(19) United States
(12) Patent Application Publication
(10) Pub. No.: US 2026/0000098 A1
(43) Pub. Date: Jan. 1, 2026

(54) CULTIVATED ANIMAL CELLS ADAPTED FOR GROWTH IN LOW AMOUNTS, AND/OR THE ABSENCE OF, DIRECT GROWTH FACTORS, INDIRECT GROWTH FACTORS, ANIMAL SERUM, AND/OR ANIMAL COMPONENTS, AND METHODS OF USE THEREOF

(71) Applicant: GOOD MEAT, INC., Alameda, CA (US)

(72) Inventors: Vitor Espirito SANTO, Alameda, CA (US); Sophia Sarah BOU-GHANNAM, Alameda, CA (US); Jungeun LEE, Alameda, CA (US)

(21) Appl. No.: 18/880,618

(22) PCT Filed: Jul. 3, 2023
(86) PCT No.: PCT/US2023/069582

§ 371 Date: Jan. 2, 2025

Related U.S. Application Data
(60) Provisional application No. 63/358,015, filed on Jul. 1, 2022.

(51) Int. Cl. A23L 13/00; C12N 5/00; C12N 5/071

(52) U.S. Cl. CPC A23L 13/00; C12N 5/0043; C12N 5/0686; C12N 2500/25; C12N 2500/46

(57) ABSTRACT

Provided herein are cultivated animal cells that are adapted to grow in growth medium that contains low-insulin or no exogenously provided insulin. Provided herein are cultivated animal cells that are adapted to grow in growth medium that contains low-transferrin or no exogenously provided transferrin. Provided herein are methods of cultivating animal cells in growth medium that contains low-insulin or no exogenously provided insulin. Provided herein are methods of cultivating animal cell in growth medium that contains low-transferrin or no exogenously provided transferrin. Also provided are food products made from cultivated animal cells.

### CROSS REFERENCE TO RELATED APPLICATIONS

This application claims benefit of and priority to U.S. Provisional Application No. 63/358,015, filed Jul. 1, 2022, which is specifically incorporated by reference herein in its entirety.

### FIELD OF THE INVENTION

Animal cell lines, adapted to grow in culture media that contains low and/or no direct growth factor, indirect growth factors, animal serum, animal components, or combinations thereof, and methods of making and using thereof, are described herein. Food products made from, or containing, such cultivated cells are also described herein.

### BACKGROUND

The consumption of animals has been a part of the human diet for thousands of years. Modern domestic cattle (Bos taurus) are believed to have been domesticated 10,000-5,000 years ago, while chickens (Gallus domesticus) are believed to have been domesticated about 8,000 years ago from the red junglefowl (Gallus gallus). Currently, it is estimated that there are between 1 billion domestic cattle and over 20 billion domestic chickens worldwide.

There is an ever-growing demand for meat to meet the needs of an increasing global population. However, conventional animal agriculture cannot address these needs effectively and sustainably.

The farming of animals for human consumption has significant environmental impacts. In 2006, the United Nations Food and Agricultural Organization estimated that animal farming produces about 18 percent of the total greenhouse gases produced by human activity, which exceeds greenhouse gases produced by the transportation industry, namely automobiles, trucks, trains, ships, and airplanes combined. Additionally, there are health risks in consuming farmed animals. The slaughter and processing of animals exposes the animal carcasses to microbial contamination and exposes people to potentially deadly microbes that remain on the meat, particularly in less-developed parts of the world which do not have the food safety and/or cold storage infrastructure necessary to properly store harvested meat.

As a result, there is an increased interest in cultured meat, also designated as cell-based meat or cultivated meat, which is produced from animal cells using cell culture methodology. Cultured meat is a sustainable alternative to traditional livestock-derived meat. Cultured meat products have the potential to: (1) substantially reduce reliance on slaughtered animals for food use; (2) lessen the environmental burden of raising animals for food supply; and (3) provide a reliable source of protein that is both safe and has consistent quality.

In conventional cell/tissue culture, insulin and transferrin are required for cell growth. Literature reports have indicated that the optimum concentration of insulin is 21.3 mg/L and the optimum concentration of transferrin is 57 mg/L. (www.pharmaceutical-technology.com/sponsored/recombinant-insulin-cell-growth-cd-media/) Similarly, in other cell types, insulin and transferrin at high concentrations are reported to be necessary for cell growth (Ghasemi, et al., Iran J Pharm Res. 2019 18 (Suppl1): 146-156. Wu, et al., PLOS ONE 14 (4): e0215022 (2019); Devireddy, et al., PLOS ONE 14 (2): e0210250 (2019); and Yao, et al., Reprod Med Biol., 16:99-177 (2017).

Insulin and transferrin are two of the more expensive ingredients used in cultivation of animal cells for food. Typically, insulin is a recombinant product and transferrin is isolated from the blood of animals. However, transferrin can also be produced using recombinant technology. Animal sera and/or animal-derived components are also typically components on cell culture media. However, the use of animal sera as a raw material may introduce batch-to-batch variation and impact negatively on the economics of large-scale cell-culture processes.

Cell culture media production for animal cells, to date, has been developed towards applications in the biopharmaceutical industry, which does not operate under the same constraints as a food production process. Moreover, the majority of the commercially available culture media are expensive and are limited by proprietary media formulations. However, the production requirements for food applications are likely to be less stringent than for therapeutic or research operations, potentially enabling cost savings resulting from the grade of raw materials and final products.

There is a need to develop an in-house defined culture media tailored to promote the growth of specific cell types and cultivation processes for cultured meat production.

There exists a need for a cost-effective culture media, which can be used to scale up production of cultivated meat.

There exists a need for a cost-effective culture media which can be used to scale up production of cultivated meat, which minimizes or eliminates direct and indirect growth factors, animal sera, and/or animal-derived components, particularly expensive indirect growth factors, such as one or more hormones and/or recombinant molecules (e.g., insulin and transferrin).

### SUMMARY

Animal cells adapted to grow in a growth medium that contains little or no direct and/or indirect growth factors are described herein. In some embodiments, the cells are adapted to grow in a growth medium that contains little or no direct and/or indirect growth factors and little or no animal serum. In some embodiments, the cells are adapted to grow in a growth medium that contains little or no direct and/or indirect growth factors, little or no animal serum, and little or no animal-derived components.

In some embodiments, the growth medium contains little or no amounts of the indirect growth factors insulin and transferrin. In some embodiments, the growth medium contains low amounts of insulin and no transferrin. In some embodiments, the growth medium contains low amounts of transferrin and no insulin. In some embodiments, the growth medium contains low amounts of insulin and transferrin. In some embodiments, the growth medium contains no insulin and no transferrin. In some embodiments, the cell culture medium contains insulin and transferring in the initial passages, but then the cells are adapted to grow in low and/or no amounts of insulin and/or transferrin over a series of passages.

In some embodiments, if the growth medium contains a low amount of insulin and/or transferrin, the amount of each of insulin and transferring is less than 50 ng/L, 45 ng/L, 40 ng/L, 35 ng/L, 30 ng/L, 25 ng/L, 20 ng/L, 15 ng/L, 10 ng/L, or 5 ng/L.

In some embodiments, the growth medium is as described above and contains low amounts or no amount of animal serum. Exemplary animal serums include, but are not limited to, calf serum and fetal bovine serum but can also include serum from other animal species. In some embodiments, low amounts of animal serum is less than about 3%, 2.9%, 2.8%, 2.7%, 2.6%, 2.5%, 2.4%, 2.3%, 2.2%, 2.1%, 2.0%, 1.9%, 1.8%, 1.7%, 1.6%, 1.5%, 1.4%, 1.3%, 1.2%, 1.1%, 1.0%, 0.9%, 0.8%, 0.7%, 0.6%, 0.5%, 0.4%, 0.3%, 0.2%, 0.1%. In some embodiments, the growth medium contains no serum. In some embodiments, the medium contains no animal serum in all stages of passaging, i.e., scale-up through production.

In some embodiments, the growth medium can contain one or more selected from fatty acids, proteins, elements, and small molecules. In some embodiments, the growth media contains selenium. In some embodiments, the growth media contains ethanolamine.

In some embodiments, the cells are immortalized cells. In some embodiments, the immortalized cells are spontaneously immortalized. In some embodiments, the immortalized cells are non-tumorigenic.

In some embodiments, the cells disclosed herein are cultivated in adherent cultures or in suspension cultures. In some embodiments, the cells disclosed herein are cultured initially in adherent cultures and then adapted to suspension culture and are cultured in suspension in production passages.

In some embodiments, one or more of the cell types or cell lines described herein have a tendency to aggregate and the cell types/cell lines are adapted to single cell in suspension.

The cell lines described herein can be any type of cell. In some embodiments, the cell type is suitable for use in the production of cultivated meat. In some embodiments, the cells are fibroblasts, kidney cells, muscle cells, myosatellite cells, myoblasts, pre-adipocytes, adipocytes, epithelial cells, or combinations thereof, of animals that are adapted for cultivation in a growth medium that is described above.

In some embodiments, fibroblasts are adapted to grow in growth medium that contains low-insulin or no exogenously provided insulin. In some embodiments, fibroblasts are adapted to grow in growth medium that contains low-transferrin or no exogenously provided transferrin. In some embodiments, fibroblasts are adapted to grow in growth medium that contains low-insulin or no exogenously provided insulin, and low-transferrin or no exogenously provided transferrin.

In some embodiments, kidney cells are adapted to grow in growth medium that contains low-insulin or no exogenously provided insulin. In some embodiments, kidney cells are adapted to grow in growth medium that contains low-transferrin or no exogenously provided transferrin. In some embodiments, kidney cells are adapted to grow in growth medium that contains low-insulin or no exogenously provided insulin, and low-transferrin or no exogenously provided transferrin.

In some embodiments, muscle cells are adapted to grow in growth medium that contains low-insulin or no exogenously provided insulin. In some embodiments, muscle cells are adapted to grow in growth medium that contains low-transferrin or no exogenously provided transferrin. In some embodiment, muscle cells are adapted to grow in growth medium that contains low-insulin or no exogenously provided insulin, and low-transferrin or no exogenously provided transferrin.

In some embodiments, fat cells, pre-adipocytes or adipocyte cells are adapted to grow in growth medium that contains low-insulin or no exogenously provided insulin. In some embodiments, fat cells, pre-adipocytes or adipocyte cells are adapted to grow in growth medium that contains low-transferrin or no exogenously provided transferrin. In some embodiments, fat cells, pre-adipocytes or adipocyte cells are adapted to grow in growth medium that contains low-insulin or no exogenously provided insulin, and low-transferrin or no exogenously provided transferrin.

In some embodiments, the various cells types described above are adapted as described above and are cultured in low amounts, or the absence of, direct growth factors, animal sera, and/or animal-derived components.

Food products containing, or made from, the cultivated animal cells described herein are also disclosed. In some embodiments, there are provided methods of producing a food product containing one or more of the cell types described above, cultured in the growth media described above. In some embodiments, the methods include culturing a population of cells in vitro in a growth medium capable of maintaining the cells, recovering cells, and formulating the recovered cells into an edible food product. In some embodiments, the cells include fibroblasts, kidney cells, muscle cells, myosatellite cells, myoblasts, fat cells, pre-adipocytes, adipocytes, epithelial cells, or combinations thereof.

In some embodiments, the method for producing a food product includes: (1) conditioning water with a phosphate to prepare conditioned water; (2) hydrating a plant protein isolate or plant protein concentrate, such as a pulse protein isolate or concentrate, with the conditioned water to produce hydrated plant protein; (3) contacting animal cells with the hydrated plant protein to produce a cell and pulse protein mixture; (4) heating the cell and plant protein mixture in steps, wherein the steps include at least one of: (i) ramping up the temperature of the cell and protein mixture to a temperature between 40-65° C.; (ii) maintaining the temperature of the cell and protein mixture at a temperature between 40-65° C. for about 1 to about 30 minutes; (iii) ramping up the temperature of the cell and protein mixture to a temperature between 60-85° C.; (iv) cooling the cell and protein mixture to a temperature between −1° C.-25° C. and (v) admixing the cell and protein mixture with a fat to create a pre-cooking product. In some embodiments, the pre-cooking product can be consumed without further cooking. Alternatively, in some embodiments, the pre-cooking product is cooked to produce the edible food product. Optionally, the pre-cooking product may be stored at room temperature, refrigeration temperatures or frozen.

In some embodiments, the food product is prepared by co-extruding a cell paste and a plant protein isolate/concentrate using an appropriate apparatus, to form a food product. In some embodiments, the apparatus can be a cooling die that has a body defining a flow path extending from an inlet at an upstream end of the body to an outlet at a downstream end of the body. In some embodiments, the inlet has a first cross-sectional area, and the outlet has a second cross-sectional area that is larger than the first cross-sectional area. Additionally, the flow path can include a transitional region at the upstream end of the body and the flow path includes a forming region extending from the transitional region to the outlet.

In some embodiments, the formulation to be extruded includes cultivated animal cells in an amount between 35-95 wt % of the formulation, and a dry mix in an amount between 25-45 wt % of the formulation. The dry mix can include a proteinaceous (e.g., plant protein) ingredient, a binding ingredient, an emulsifier, and one or more flavorants.

In some embodiments, there are provided food products contain cells of the genus Bos or genus Gallus, the food product containing a cell paste at a content of at least 5% by weight; a plant protein isolate or plant protein concentrate, the plant protein content being at least 5% by weight of the food product; a fat, the fat content being at least 5% by weight of the food product; and water, the water content being at least 5% by weight of the food product.

In some embodiments, the food composition or food product contains about 1%-100% by weight wet cell paste.

In some embodiments, plant protein isolates or plant protein concentrates are obtained from pulses selected from dry beans, lentils, mung beans, fava beans, dry peas, chickpeas, cowpeas, bambara beans, pigeon peas, lupins, vetches, adzuki, common beans, fenugreek, long beans, lima beans, runner beans, or tepary beans, soybeans, mucuna beans, or combinations thereof.

In some embodiments, the pulse protein isolate/concentrate is a soybean isolate/concentrate.

In some embodiments, the pulse protein isolates or plant protein concentrates provided herein are derived from Vigna angularis, Vicia faba, Cicer arietinum, Lens culinaris, Phaseolus vulgaris, Vigna unguiculata, Vigna subterranea, Cajanus cajan, Lupinus sp., Vetch sp., Trigonella foenum-graecum, Phaseolus lunatus, Phaseolus coccineus, or Phaseolus acutifolius. In some embodiments, the pulse protein isolates are derived from mung beans. In some embodiments, the mung bean is Vigna radiata.

In some embodiments, animal protein isolate and animal protein concentrate are obtained from animals or animal products. Examples of animal protein isolate or animal protein concentrate include collagen, whey, casein, and egg protein.

In some embodiments, plant protein isolates are obtained from wheat, rice, teff, oat, corn, barley, sorghum, rye, millet, triticale, amaranth, buckwheat, quinoa, almond, cashew, pecan, peanut, walnut, macadamia, hazelnut, pistachio, brazil, chestnut, kola nut, sunflower seeds, pumpkin seeds, flax seeds, cacao, pine nut, ginkgo, and other nuts.

### BRIEF DESCRIPTIONS OF THE DRAWINGS

FIG. 1 shows the viable cell densities of chicken cells cultivated in growth medium that contains 100% insulin and transferrin (solid line) as well as the viable cell densities of chicken cells cultivated in growth medium that contains no exogenously provided insulin and no exogenously provided transferrin (dashed line).

FIG. 2 shows the viable cell densities of bovine cells cultivated in growth medium containing various amounts of insulin and transferrin (percentages above the cell densities) as function of culture step.

FIGS. 3A and 3B show the viable cell densities of avian cells cultivated in bioreactors in growth medium that contains 100% insulin and transferrin (solid lines) as well as the viable cell densities of avian cells cultivated in growth medium that contains no exogenously provided insulin and no exogenously provided transferrin (dashed lines).

FIGS. 4A-4B are graphs showing: the population double level (PDL) as a function of time (days) (FIG. 4A) and cell viability as a function of passage number (FIG. 4B) for an adherent expanded bovine cell line.

FIG. 5 is a graph showing viable cell density as a function of culture time (days) for a bovine cell line in suspension cultivation.

FIG. 6 is a graph showing cell viability (%) as a function of passage number for a bovine cell line in suspension cultivation.

FIG. 7A is a graph showing population doubling level, as a function of time (days) (.

FIG. 7B is a bar graph showing population doubling times (PDT) as a function of passage number. FIG. 7C is a bar graph showing cell viability (%) as a function of passage number in suspension culture for SFC7 and MEM-bactone cultures for a bovine cell line.

FIG. 8 shows pictographic depictions of the three approaches used for single cell adaptation for a bovine cell line in suspension cultivation. The Control condition uses enzymatic dissociation; the single cell adaptation Scheme 1 and Scheme 2 implement size-exclusion selection without enzymatic dissociation.

FIG. 9A is a graph showing the viable cell density as a function of culture time (days). FIG. 9B is a graph showing population doubling time as a function of passage number. FIG. 9C is a graph showing cell (%) as a function of passage number in for a bovine cell line experiencing Scheme 2 single cell adaptation in suspension culture with chemically defined, animal-free media. The annotation “SC” indicates the point at which cells achieved a predominantly single cell population. The annotation “split adapt” indicates the timeframe over which single cells were adapted to a passaging style that continuously carries over a portion of spent media.

### DETAILED DESCRIPTION

The following description is presented to enable one of ordinary skill in the art to make and use the disclosed subject matter and to incorporate it in the context of applications. Various modifications, as well as a variety of uses in different applications, will be readily apparent to those skilled in the art, and the general principles defined herein may be applied to a wide range of embodiments. Thus, the present disclosure is not intended to be limited to the embodiments presented but is to be accorded the widest scope consistent with the principles and novel features disclosed herein.

### I. Definitions

As used herein, the term “direct growth factor” refers to molecules that directly stimulate cell proliferation and differentiation. Growth factors are specific to cell types due to the expression of highly specific cell surface receptors. Examples include, but are not limited to, insulin growth factor (IGF), vascular endothelial growth factor (VEGF), epidermal growth factor (EGF), and platelet-derived growth factor (PDGF). “Growth Factor” also includes undefined, complex mixtures derived from biological extracts and lysates that contain growth factors and can be used as a cell culture media supplement to directly support cell proliferation, survival, and differentiation. This includes, but is not limited to, blood-derived, blood plasma-derived, and blood component-derived extracts, including but not limited to serum and platelet-lysates from fetal and adult sources, as well as complex mixtures from tissue extracts and biological fluids, including but not limited to embryonic extracts, pituitary extracts, bone marrow, placental or cord serum, plasma clots, amniotic fluid, colostrum, and mature milk. Direct growth factors can be isolated from natural sources or can be prepared using recombinant techniques known in the art.

As used herein, the term “indirect growth factor” refers to molecules that indirectly affect or influence cell proliferation and differentiation. Indirect growth factors can be isolated from natural sources or can be prepared using recombinant techniques known in the art. Examples include, but are not limited to, hormones, such as insulin or insulin-like hormone; and cell transport molecules, such as transferrin.

As used herein, the term “batch culture” refers to a closed culture system with nutrient, temperature, pressure, aeration, and other environmental conditions to optimize growth. Because nutrients are not added, nor waste products removed during incubation, batch cultures can complete a finite number of life cycles before nutrients are depleted and growth stops.

As used herein, the term “fed-batch culture” refers to a culture system in which at a desired time a portion of the cells in the culture medium is removed (harvested) and fresh culture medium is added to the cell culture system, for example, a bioreactor, in full or partial replacement of the harvested cells. With the addition of fresh nutrients and removal of waste products, cells in the fed-batch cultures can continue to multiply and be harvested. Fed-batch cultures can be maintained for a long time or an indefinite amount of time so long as the bioreactor does not become contaminated with adventitious organisms. As used herein, the term “fed-batch culture” also refers to an operational technique where one or more nutrients, such as substrates, are fed to a bioreactor in continuous or periodic mode during cultivation and in which product(s) remain in the bioreactor until the end of a run. An alternative description is that of a culture in which a base medium supports initial cell culture and a feed medium is added to prevent nutrient depletion. In a fed-batch culture one can control concentration of fed-substrate in the culture liquid at desired levels to support continuous growth.

As used herein, the term “edible food product” refers to a food product safe for human consumption. For example, this includes, but is not limited to a food product that is generally recognized as safe per a government or regulatory body (such as the United States Food and Drug Administration). In certain embodiments, the food product is considered safe to consume by a person of skill. Any edible food product suitable for a human consumption should also be suitable for consumption by another animal and such an embodiment is intended to be within the scope herein.

As used herein, the term “enzyme” or “enzymatically” refers to biological catalysts. Enzymes accelerate, or catalyze, chemical reactions. Enzymes increase the rate of reaction by lowering the activation energy.

As used herein, the term “expression” is the process by which information from a gene is used in the synthesis of a functional gene product. As used herein, the term “endogenously expressed” means that the cell expresses a gene that is naturally present in the cell without genetic manipulation.

As used herein, the term “downregulated” means that the expression of a gene in a cell is decreased. For example, when myosatellite cells are differentiated or activated into muscle cells or myoblasts, the expression of certain genes is downregulated in the muscle cells or the myoblasts as compared to myosatellite cells. Similarly, for example, when preadipocytes differentiate into fat cells or adipocytes, the expression of certain genes is downregulated in the fat cells or adipocytes as compared to preadipocyte cells. Similarly mesenchymal cells that develop into fibroblasts will downregulate the expression of certain genes in the mature fibroblast.

As used herein, the term “upregulated” means that the expression of a gene in a cell is increased. For example, when myosatellite cells are differentiated or activated into muscle cells or myoblasts, the expression of certain genes is upregulated in the muscle cells or the myoblasts as compared to myosatellite cells. Similarly, for example, when preadipocytes differentiate into fat cells or adipocytes, the expression of certain genes is upregulated in the fat cells or adipocytes as compared to preadipocyte cells. Similarly mesenchymal cells that develop into fibroblasts will upregulate the expression of certain genes in the mature fibroblast.

As used herein “heterologous” means having a different relation, relative position, or structure. Thus, unless otherwise specified, heterologous includes joining or linking of two or more amino acid or nucleic acid sequences from that organism (e.g., species) that are not normally found joined or linked (e.g., together) as well as joining or linking of two or more amino acid or nucleic acid sequences from different species.

As used herein “endogenous” refers to any material from or produced inside an organism, cell, tissue or system including but limited to a cell culture system.

As used herein “exogenous” refers to any material introduced from or produced outside an organism, cell, tissue or system including but not limited to a cell culture system.

As used herein, the term “exogenous expression,” “exogenously expressed,” or the like also means that a gene that is not naturally present in an un-engineered cell (host cell) is expressed in the host cell by introducing one or more copies of a recombinant gene into the host cell. As used herein, the term “exogenous expression,” “exogenously expressed,” or the like also means that a gene that is naturally present in an un-engineered cell (host cell) is expressed in a host cell by introducing one or more copies of a recombinant gene into the host cell.

As used herein, the term “knock-in” refers to an engineered cell, or a method to produce an engineered cell, in which an exogenous gene is introduced into the host cell.

As used herein, the term “knock-out” refers to an engineered cell, or a method to produce an engineered cell, in which a gene that is naturally present in the host cell is (endogenous gene) is deleted or altered in a manner to prevent or reduce expression of the endogenous gene.

As used herein, “fibroblasts” refer to mesenchymal-derived cells that are responsible for the extracellular matrix, epithelial cell differentiation, and regulation of inflammation and wound healing. In addition, fibroblasts are also responsible for the secretion of growth factors and work as scaffolds for other cell types. Fibroblasts are one cell type found in conventional meat. Fibroblasts cells are cells wherein the cell marker is selected from the group consisting of ACTA2 (actin Alpha 2), FAP (Fibroblast activation protein-α), PDGFRB (platelet derived growth factor receptor beta), S100A4 (S100 Calcium Binding Protein A4), FN1 (Fibronectin 1), COL1A1 (collagen, type I, alpha 1), POSTN (Periostin), DCN (decorin), FBLN2 (Fibulin 2), COL1A2 (collagen, type I, alpha 2).

As used herein “weaned” cells and “adapted cells” are cells in which the cells have been weaned or adapted to grow in culture media that does not include an exogenously added nutrient, such as insulin and/or transferrin.

As used herein, the term “myosatellite cell” is a muscle stem cell that is multipotent and can differentiate into mature muscle cells. Myosatellite cells are cells wherein the endogenous expression of a cell marker gene product is selected from the group consisting of CxCL4 (chemokine (C—X—C motif) ligand 4), Spry 1 (sprouty RTK signaling antagonist 1), CD56, PAX7 (Paired Box 7) and PAX3 (Paired Box 3).

As used herein, the term “muscle cell” or “myoblast” is a cell that is not a myosatellite cell. Muscle cells or myoblasts are cells in which the endogenously expressed cell markers are selected from the group consisting of MyoD (myogenic differentiation 1), Myf5 (Myogenic factor 5), HGF (Hepatocyte Growth Factor), FGF2 (Fibroblast growth factor 2), CD56 (also known as neural cell adhesion molecule 1).

As used herein, the term “fat cells” or “adipocytes” are cells that specialize in storing energy as fat. Fat cells or adipocytes are cells wherein the expression of cell markers are selected from the group consisting of Adiponectin, lipoprotein lipase, perilipin, leptin, and FABP4 (fatty acid binding protein 4).

As used herein, the term “preadipocytes” or “fat stem cells” are cell capable of differentiating into fat cells or adipocytes. Preadipocytes or fat stem cells are cells wherein the expression of the cell markers are selected from the group consisting of PPAR gamma (PPARγ), CEBP alpha (CEBPα), SREBP (Sterol regulatory element binding protein), Zfp423 (zinc finger protein 423), GATA3 (GATA Binding Protein 3), Wnt10b (Wnt Family Member 10B), Wnt10a (Wnt Family Member 10A), Wnt6 (Wnt Family Member 6), Mmp3 (Matrix metalloprotease 3), and Twist2 (twist family bHLH transcription factor 2).

As used herein, kidney or renal cells are epithelial cells wherein the expression of cell markers are selected from the group consisting of ACTA2, FAP, PDGFRA (platelet derived growth factor receptor alpha), PDGFRB, S100A4, FN1, COL1A1, POSTN, DCN, FBLN2, COL1A2, DES, and CDH11 (cadherin 11).

As used herein, epithelial cells are cells wherein the expression of cell markers are selected from the group consisting of EPCAM (epithelial cellular adhesion molecule), CDH1 (E-Cadherin), KRT7 (Keratin 7), KRT8 (Keratin 8), KRT18 (Keratin 18), and KRT19 (Keratin 19).

As used herein, Lung epithelial cells are cells that endogenously express the cell surface markers selected from the group consisting of Foxa1 (forkhead box A1), KRT5 (Keratin 5), and KRT14 (Keratin 14).

As used herein, Renal epithelial cells are cells that endogenously express the cell surface markers selected from the group consisting of K7 (keratin 7), K8 (keratin 8), K18 (keratin 18), and K19 (keratin 19).

As used herein, Intestinal epithelial cells are cells that endogenously express the cell surface markers selected from the group consisting of MUC17 (mucin 17), CDH17 (cadherin 17), and CEACAM7 (CEA cell adhesion molecule 7).

As used herein, the term “non-tumorigenic” means a cell that does not express a family of genes that belong to pathways described to trigger formation or growth of tumors, including but not limited to pathways implicated in cancer (KEGG_05200), transcriptional misregulation in cancer (KEGG_05202), microRNAs in cancer (KEGG_05206), proteoglycans in cancer (KEGG_05205), chemical carcinogenesis (KEGG_05204), viral carcinogenesis (KEGG_05203), central carbon metabolism in cancer (KEGG_05230), choline metabolism in cancer (KEGG_05231) and PD-L1 expression and PD-1 checkpoint pathway in cancer (KEGG_05235).

As used herein, the term “immortalized cell” is a cell that can be propagated in vitro for more than 60 population doublings and in the case of some cell lines, they can be propagated indefinitely.

As used herein a “cell marker” is a protein that is expressed by the cells. Cell types of different lineages express different cell markers.

As used herein, “desmin” and “myosin” are proteins expressed by committed and/or differentiated muscle cell. “Myosin heavy chain 2” (MyHC2) is a fibrous protein that is expressed by a differentiated muscle cell.

As used herein, an “animal” is captive bred and cultivated or harvested from the wild for consumption. Animals that are commonly captive bred and consumed by humans include vertebrates, invertebrates, and insects. Examples of vertebrates include chicken, duck, turkey, goose, quail, pigeon ostrich, cow, pig, lamb, goat horse, rabbit and fish, Examples of invertebrates include crustaceans such as shrimp, crab, lobster, crayfish octopus, squid, oyster, mussel, and snail.

As used herein, a bird of the genus Gallus is an animal that is farmed for human consumption. Gallus gallus is the common domesticated chicken.

As used herein, a bovine of the Bos genus is an animal that is farmed for human consumption. Species of Bos include B. buiaensis, B. frontails, B. grunniens, B. javanicus, B. savueli, and B. taurus.

As used herein, an animal of the genus Sus, is commonly referred to as a pig. The domestic pig is Sus scrofa domesticus.

As used herein the term “small molecule” is a molecule that has a molecular weight of less than 5,000 Dalton.

As used herein, a “gene product” is the biochemical material, either RNA or protein, resulting from expression of a gene.

As used herein, “growth medium” refers to a medium or culture medium that supports the growth of microorganisms or cells or small plants. A growth medium may be, without limitation, solid or liquid or semi-solid. Growth medium shall also be synonymous with “growth media.”

As used herein, “basal medium” refers to a non-supplemented medium which promotes the growth of many types of microorganisms and/or cells which do not require any special nutrient supplements.

As used herein, “in vitro” refers to a process performed or taking place in a test tube, culture dish, bioreactor, or elsewhere outside a living organism. In the body of this disclosure, a product may also be referred to as an in vitro product, in which case in vitro shall be an adjective and the meaning shall be that the product has been produced with a method or process that is outside a living organism.

As used herein the term “insulin” refers to the hormone insulin or insulin-like hormone.

As used herein, the term “low-insulin” refers to a concentration of insulin that is less than or equal to 50 ng/L.

As used herein, the term “no exogenously provided insulin” or “no exogenously added insulin” refers to cultivation conditions or growth media in which no insulin is provided. During production of cultivated animal cells in large bioreactors the seed train and the production cultivation run are cultivated in growth media that is not provided with insulin.

As used herein a “production cultivation run” is the final cultivation step in the production of cultivated meat. The inoculum of a production cultivation run is prepared by preparing and performing a seed train in which a small inoculum is cultivated in successively larger cultivation volumes. As a non-limiting example, the first step in a seed train will often start with about 1 mL frozen cells called a working cell bank (WCB) and expanded to a much larger volume in a series of cultivation steps. Typically, a 1.5 mL vial, containing 1 mL of WCB, is thawed and cultivated in a volume larger than 1 mL, typically 50 mL. After a desired cell density in the first seed train step is reached, the cells from the first seed train step are expanded to a larger volume in growth medium. The inoculum volume of all seed train steps, after the first seed train cultivation step is typically 20%-40% of a desired seed train volume. For example, a 50 mL inoculum can be used to inoculate a seed train cultivation volume of 250 mL. Upon reaching a desired cell density, the 250 ml volume is used as an inoculum in a 1 L seed train cultivation volume. Additional seed train steps may be repeated one or more times to obtain cells at sufficient density and volume to inoculate the production cultivation run. For example, for a production cultivation run at 10,000 L scale, the inoculum is between 2000 L-4000 L.

As used herein the term “transferrin” refers to a protein that transports iron through blood plasma.

As used herein, the term “low-transferrin” refers to a concentration of transferrin that is lower than 50 ng/L

As used herein, the term “no exogenously provided transferrin” or “no exogenously added transferrin” refers to cultivation conditions or growth media in which no transferrin is provided. As a non-limiting example, during production of cultivated animal cells scale in large bioreactors that are greater than 1,000 L, the seed train of the cultivated animal cells may be cultivated in growth media that is provided with transferrin. A production cultivation in a large bioreactor with no exogenously provided transferrin is a cultivation run in which the final seed train step may be performed with exogenously provided transferrin but the production cultivation run is performed with no exogenously added transferrin. As an example, the first step in a seed train run will often start with about 1 mL frozen cells. The frozen cells are thawed and cultivated in a volume larger than 1 mL in which exogenous transferrin is provided. After a desired cell density is reached, the cells from the first seed train step are expanded to a larger volume in growth medium with exogenously provided transferrin. The seed train steps may be repeated one or more times to obtain cells at sufficient density and volume to inoculate the production cultivation run. Typically, the inoculum of the production cultivation run is about 20% of the final volume of the production cultivation run. For example, for a production cultivation run at 1,000 L scale, the inoculum is about 200 mL.

As used herein, “master cell bank” or “MCB” refers to cells produced from an original cell line that are cryopreserved. The original cell line is often identified as a “research cell bank” or “RCB” and are also typically cryopreserved. MCBs are prepared by taking a vial of the RCB and expanding the RCB cells by cultivating the cells and freezing the cells into multiple vials.

As used herein, “working cell bank” or “WCB” refers to cells produced by taking a vial of MCB and expanding the MCB cells by cultivating the cells and freezing the cells into multiple vials. The WCBs are used as the inoculum in a seed train for a production cultivation run.

As used herein, “suspension culture” refers to a type of culture in which single cells or small aggregates of cells multiply (grow) while suspended in agitated liquid medium. It also refers to a cell culture or a cell suspension culture.

As used herein, “adherent culture” refers to a type of culture in which cells can propagate or multiply (grow) while adhered to the surface of a flask or other scaffold. The scaffold is any object that provides a surface on to which the cells adhere. The scaffold can be an edible object, for example but not limited to an extruded protein or an extruded cell.

As used herein, “cell paste” refers to a paste of cells harvested from a cell culture that contains water. The dry cell weight of cell paste may be 1%-5%, 5%-10%, 10%-15%, 15%-20%, 20%-25%, 25%-30%, 30%-35%, 35%-40%, 40%-45%, 45%-50%, or higher. A skilled worker can prepare cell paste with a desired water content. Typically, cell paste contains about 5%-15% cells by dry cell weight. It is within the ambit of skilled practitioners to prepare cell paste that contains a desired dry cell weight of cultivated cells, including cell paste that contains any other desired percentage by dry cell weight. The skilled worker can remove moisture by centrifugation, lyophilization, heating or any other well-known drying techniques. According to the United States Department of Agriculture, the naturally occurring moisture content of animal meats including beef, is about 75% water. In some embodiments, the cell paste provided herein contains a significant amount of water (i.e., wet cell paste). “Wet cell paste” as used herein contains about 25%-90% water 25%-85% water, 25%-80% water, 25%-75% water, 25%-70% water, 25%-65% water, 25%-60% water, 25%-55% water, 25%-50% water, 30%-90% water, 30%-85% water, 30%-80% water, 30%-75% water, 30%-70% water, 30%-65% water, 30%-60% water, 30%-55% water, 30%-50% water, 35%-90% water, 35%-85% water, 35%-80% water, 35%-75% water, 35%-70% water, 35%-65% water, 35%-60% water, 35%-55% water, 35%-50% water, 40%-90% water, 40%-85% water, 40%-80% water, 40%-75% water, 40%-70% water, 40%-65% water, 40%-60% water, 40%-60% water, 40%-55% water, 40%-50% water, 45%-90% water, 45%-85% water, 45%-80% water, 45%-75% water, 45%-70% water, 45%-75% water, 45%-70% water, 45%-65% water, 45%-60% water, 45%-55% water, 45%-50% water, 50%-90% water, 50%-85% water, 50%-80% water, 50%-75% water, 50%-70% water, 50%-65% water, 50%-60% water, or 50%-55% water. Cell paste is another term for cultured cell meat.

As used herein, “substantially pure” refers to cells that are at least 80% cells by dry weight. Substantially pure cells are between 80%-85% cells by dry weight, between 85%-90% cells by dry weight, between 90%-92% cells by dry weight, between 92%-94% cells by dry weight, between 94%-96% cells by dry weight, between 96%-98% cells by dry weight, between 98%-99% cells by dry weight.

As used herein, “seasoning” refers to one or more herbs and spices in both solid and liquid form.

As used herein, “primary cells” refer to cells from a parental animal that maintain growth in a suitable growth medium, for instance under controlled environmental conditions. Cells in primary culture have the same karyotype (number and appearance of chromosomes in the nucleus of a eukaryotic cell) as those cells in the original tissue.

As used herein, “secondary cells” refers to primary cells that have undergone a genetic transformation and become immortalized allowing for indefinite proliferation.

As used herein, “proliferation” refers to a process that results in an increase in the number of cells. It is characterized by a balance between cell division and cell loss through cell death or differentiation.

As used herein, “adventitious” refers to one or more contaminants such as, but not limited to: viruses, bacteria, mycoplasma, and fungi.

As used herein “peptide cross-linking enzyme” or “cross-linking enzyme” is an enzyme that catalyzes the formation of covalent bonds between one or more polypeptides.

As used herein, “transglutaminase” or “TG” refers to an enzyme (R-glutamyl-peptide amine glutamyl transferase) that catalyzes the formation of a peptide (amide) bond between γ-carboxyamide groups and various primary amines, classified as EC 2.3.2.13. Transglutaminases catalyze the formation of covalent bonds between polypeptides, thereby cross-linked polypeptides. Cross-linking enzymes such as transglutaminase are used in the food industry to improve texture of some food products such as dairy, meat and cereal products. Cross-linking enzymes can be isolated from a bacterial source, a fungus, a mold, a fish, a mammal, or a plant.

As used herein “protein concentrate” is a collection of one or more different polypeptides obtained from a plant source or animal source. The percent protein by dry weight of a protein concentrate is greater than 25% protein by dry weight.

As used herein “protein isolate” is a collection of one or more different polypeptides obtained from a plant source or an animal source. The percent protein by dry weight of a protein concentrate is greater than 50% protein by dry weight.

As used herein, and unless otherwise indicated, percentage (%) refers to total % by weight typically on a dry weight basis.

The term “about” indicates and encompasses an indicated value and a range above and below that value. In certain embodiments, the term “about” indicates the designated value ±10%, ±5%, or ±1%. In certain embodiments, the term “about” indicates the designated value ±one standard deviation of that value.

In this disclosure, methods are presented for culturing Bos taurus cells in vitro. The methods herein provide methods to proliferate, recover, and monitor the purity of cell cultures. The cells can be used, for example, in one or more food products.

It is to be understood that the disclosed method and compositions are not limited to specific synthetic methods, specific analytical techniques, or to particular reagents unless otherwise specified, and, as such, can vary. It is also to be understood that the terminology used herein is for the purpose of describing particular embodiments only and is not intended to be limiting.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

Throughout this specification the word “comprise,” or variations such as “comprises” or “comprising,” will be understood to imply the inclusion of a stated element, integer or step, or group of elements, integers or steps, but not the exclusion of any other element, integer or step, or group of elements, integers or steps.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a ligand is disclosed and discussed and a number of modifications that can be made to a number of molecules including the ligand are discussed, each and every combination and permutation of ligand and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials.

These concepts apply to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

All methods described herein can be performed in any suitable order unless otherwise indicated or otherwise clearly contradicted by context. The use of any and all examples, or exemplary language (e.g., “such as”) provided herein, is intended merely to better illuminate the embodiments and does not pose a limitation on the scope of the embodiments unless otherwise claimed. No language in the specification should be construed as indicating any non-claimed element as essential to the practice of the invention.

Unless otherwise indicated, the disclosure encompasses conventional techniques of molecular biology, microbiology, cell biology and recombinant DNA, which are within the skill of the art. Unless otherwise noted, technical terms are used according to conventional usage, and in the art, such as in the references cited herein, each of which is specifically incorporated by reference herein in its entirety.

### II. Cells

Provided herein are cultivated animal cells adapted for growth in growth medium that contains little or no direct and/or indirect growth factors, animal serum, animal-derived components, or combinations thereof. In some embodiments, the growth medium contains little or no direct growth factors and little or no indirect growth factors insulin and transferrin. In some embodiments, the growth medium contains little or no animal serum and/or animal-derived components.

In some embodiments, the animal cells are adapted to grow in growth media that contains less than about 10 ng/L, less than 5 ng/L, less than 1 ng/L, less than 0.5 ng/L, or less than 0.1 ng/L insulin.

In some embodiments, the animal cells are adapted to grow in growth media that contains less than about 10 ng/L, less than 5 ng/L, less than 1 ng/L, less than 0.5 ng/L, or less than 0.1 ng/L transferrin.

### A. Bovine Cell Lines

In some embodiments, the cells are Bos taurus cells. In some embodiments, the cells are selected from, but not limited to, Bos taurus breeds: Angus, Charolais, Hereford, Simmental, Longhorn, Gelbvieh, Holstein, Limousin, Highlands, and Wagyu. In some embodiments, the cells contains/are primary Bos taurus cells. In some embodiments, the cells contains/are secondary Bos taurus cells.

In some embodiments, the cell line contains/is an epithelial cell line. Cultivated meat typically involves the use of skeletal muscle cell lines. However, such cell lines can be difficult to grow in suspension as single cells; exhibit poor proliferation; and/or are difficult to immortalize.

In some embodiments, the cell line contains/is a bovine epithelial cell line. In some embodiments, the cell line contains/is a bovine renal (kidney) cell line. In some embodiments, the bovine kidney cell line contains/is MDBK (Madin-Darby bovine kidney) cells. MDBK cells are immortalized, can be grown in suspension as single cells, and are highly scalable. MDBK cells also has the additional benefit of being non-genetically modified (i.e., are non-GMO). Thus, in some embodiments, the bovine kidney cells are not transformed or tranfected with exogenous or/and heterologous nucleic acids.

In some embodiments, the epithelial cell lines as described above are cultured as described above. In some embodiments, the epithelial cell lines described above are cultured in conventional media containing standard/conventional amounts of insulin and/or transferrin, direct growth factors, animal serum, animal derived components, and combinations thereof. A non-limiting example of such standard/conventional amounts are those amount discussed herein prior to the initiation of I/T weening. The workings examples herein, e.g., Example 2, Example 7, etc., provide examples of culturing bovine cells with standard/conventional amounts of insulin and/or transferrin, reduced levels of insulin and/or transferring, low levels of insulin and/or transferrin, and no insulin and/or transferrin.

The disclosure herein sets forth embodiments for food products compositions containing Bos taurus cells grown in vitro. In some embodiments, the compositions contain plant protein, cell paste, fat, water, and a peptide cross-linking enzyme.

### B. Avian Cell Lines

In some embodiments, the cell line contains/is an avian cell line. In some embodiments, the avian cells are selected from, but not limited to: chicken, pheasant, goose, swan, pigeon, turkey, and duck. In some embodiments, the cells are Gallus cells. In some embodiments, the cells are selected from, but not limited to, domesticated chicken.

In some embodiments, the avian cell line contains/is an avian fibroblast cell line. In some embodiments, the avian fibroblast cell line contains/is primary avian fibroblast cells. In some embodiments, the avian fibroblast cell line contains/is secondary avian fibroblast cells.

In some embodiments, the cells are UMNSAH/DF1 (C1F) cells. In certain embodiments, the cells are a commercially available chicken cell line deposited at American Type Culture Collection (ATCC, Manassas, Virginia, USA) on Oct. 11, 1996. In some embodiments, the cells used are derived from ATCC deposit number CRL12203.

### III. Culture Media and Cell Growth

A. Adaption of Cells to Low and/or No Direct Growth Factors and/or Indirect Growth Factors

Animal cells adapted to grow in a growth medium that contains little or no direct and/or indirect growth factors are described herein. In some embodiments, the cells are adapted to grow in a growth medium that contains little or no direct and/or indirect growth factors and little or no animal serum. In some embodiments, the cells are adapted to grow in a growth medium that contains little or no direct and/or indirect growth factors, little or no animal serum, and little or no animal-derived components.

In some embodiments, cell proliferation or growth occurs in suspension or adherent conditions, with or without feeder-cells, and/or in low-insulin containing growth media or growth media that does not contain exogenously provided insulin.

In some embodiments, cell proliferation or growth occurs in suspension or adherent conditions, with or without feeder-cells and/or in low-transferrin containing growth media or growth media that does not contain exogenously provided transferrin.

In some embodiments, cell proliferation or growth occurs in suspension or adherent conditions, in low-insulin containing growth media or growth media that does not contain exogenously provided insulin.

In some embodiments, cell proliferation or growth occurs in suspension or adherent conditions, in low-transferrin containing growth media or growth media that does not contain exogenously provided transferrin.

In some embodiments, cell proliferation or growth occurs in suspension or adherent conditions, in low-insulin containing growth media or growth media that does not contain exogenously provided insulin and in low-transferrin containing growth media or growth media that does not contain exogenously provided transferrin.

In some embodiments, cell proliferation or growth occurs in suspension or adherent conditions, in growth media that contains low-insulin and low-transferrin.

In some embodiments, cell proliferation or growth occurs in suspension or adherent conditions, in growth media that contains no exogenously provided insulin and no exogenously provided transferrin.

In some embodiments, the cells are cultured in a medium as described above and contain low, or no, amounts of direct growth factors, animal serum, animal-derived components, and combinations thereof.

In some embodiments, the cell lines described here are adapted to culture media contain low amounts of, or no, insulin or transferrin as described above. In some embodiments, the cell culture media is weaned of exogenous insulin and/or transferrin. In some embodiments, the cell lines grown in low or no insulin and/or transferrin had no compromise in cell growth and exhibited growth parameters comparable to cells requiring insulin and transferrin in the growth media.

In some embodiments, concentrations of insulin and transferrin were gradually reduced from the culture media. In some embodiments, once insulin and transferrin are completely removed from the growth media, cell adaptation is initiated by spin passage using growth media without the addition of insulin and transferrin. When cell growth is stabilized with consistent cell growth, research cell banks can be generated.

In some embodiments, the cells are adapted to grow in low or no amounts of insulin and/or transferrin. This process can also be described as weaning insulin and/or transferrin from the growth medium or weaning cells from insulin and/or transferrin.

In some embodiments, the cells undergo a gradual reduction of exogenously provided insulin and transferrin supplementation relative to the fully available levels of insulin and transferrin in a chemically defined maintenance media formulation, otherwise defined as insulin/transferrin (I/T) weaning.

In some embodiments, to do so, a continuous cultivation of cells is maintained with progressive, stepwise reduction of I/T to enable the cells to adapt to growth and survival in these conditions. Additionally, this gradual approach allows for any carry over effects of I/T availability in previous passages to progressively recede, so that by the final phases of I/T removal, the cells are fully adapted to low/no availability of I/T.

In some cell types, an immediate removal of I/T may see little or no change in cell performance in one to several subsequent passages due to a carryover effect of previous I/T supplementation; however, in some cell types, this approach causes a near-term and acute diminishment in cell performance within several passages due to acute removal of I/T without time for mechanistic adaptation.

In some embodiments, the weaning schedule is stepwise, with the number of steps varying by the level of cell sensitivity to the reduction of I/T. For instance, some cell types can tolerate significant reductions in I/T supplementation, in which case the 100% I/T condition was overfeeding; some cell types may require more narrow reduction intervals if cell performance suffers substantially in response to adaptation to reduced IT, in which case the 100% I/T condition was feeding more precisely. The table below outlines a general I/T weaning schedule.

| | Exogenously available | Exogenously available | | |
|---|---|---|---|---|
| | insulin as a | transferrin as a | | |
| | proportion of the | proportion of the | | Duration |
| | concentration | concentration | | of |
| | available in | available in | | adaptation |
| Step | maintenance media | maintenance media | Notes | step |
| 1 | 100% | 100% | Insulin and transferrin concentrations in | 0-5 passages; 1-20 days |
| | | | maintenance media (full insulin and | |
| | | | transferrin) | |
| 2 | 99%-60% | 99%-60% | 1-40% reduction of insulin and | 0-5 passages; 1-20 days |
| | | | transferrin | |
| 3 | 90%-40% | 90%-40% | 10-60% reduction of insulin and | 0-5 passages; 1-20 days |
| | | | transferrin | |
| | 80%-20% | 80%-20% | 20-80% reduction of insulin and | 0-5 passages; 1-20 days |
| | | | transferrin | |
| 5 | 60%-10% | 60%-10% | 40-90% reduction of insulin and | 0-5 passages; 1-20 days |
| | | | transferrin | |
| 6 | 30%-1% | 30%-1% | 70-99% reduction of insulin and | 0-5 passages; 1-20 days |
| | | | transferrin | |
| 7 | 0% | 0% | No exogenously provided insulin and | 0-5 passages; 1-20 days |
| | | | transferrin | |

As shown in the non-limiting exemplary weaning schedule above, the cell culture is started at step 1, with 100% supplementation of the exogenous I/T available in a standard maintenance media. Within 0-5 passages of stable suspension cultivation in Step 1 conditions, the cells are collected and passaged in a way that the 100% I/T media is completely removed (otherwise known as spin passaging) and then exchanged for media with a reduction, e.g., 20%, in I/T supplementation. The culture goes through another number, e.g. 0-5, of passages in this Step 2 condition and is monitored closely for growth and survival performance, primarily using metrics such as PDT, PDL, and viability. When performance achieves a threshold that is within specification for the expected performance of these cells in the maintenance media condition (i.e., 100% I/T), the culture is considered adapted to the reduced I/T level and the next step in insulin and transferrin reduction is performed.

In some embodiments, weaning is performed in this way for all subsequent reduction steps until the complete removal of insulin and transferrin is achieved. Cells are maintained for a number, e.g., 0-5, of passages in media with no exogenously provided I/T with monitoring of culture performance. When stable performance is achieved within the threshold of expected performance with 100% IT, the culture is considered adapted for growth in the absence of insulin and transferrin.

In some embodiments, the cells are repeatedly split-passaged in the presence of insulin and transferrin. In some embodiments, the cells are split-passaged less than about 10, 9, 8, 7, 6, or 5 passages. In some embodiments, the number of split-passages is less than about 5. In some embodiments, the number of split-passages is about 3.

In some embodiments, cell adaptation to the media without exogenously provided insulin and transferrin was performed with a spin passage at a targeted inoculation density of ˜0.5 million cells/mL. In some embodiments, the total number of passages performed to wean the cells from insulin and transferrin was less than about 20, 19, 18, 17, 16, 15, 14, 13, 12, 11, 10, 8, 6, or 4. In some embodiments, the number of passages was less than about 10. In some embodiments, the number of passages is from about 6 to about 10. In some embodiments, the nature of each passage is defined in a specific adaptation schedule. An exemplary, non-limiting, adaptation schedule is shown in Table 2 in the Examples.

In some embodiments, cell adaptation is initiated by removing a commercially available reagent that contains insulin and transferring, and optionally one or more additional components. In some embodiments, the commercially available reagent is ITSX (for example from ThermoFisher Scientific, catalog number 51500056), which contains insulin, transferrin, sodium selenite, and ethanolamine. In some embodiments, the desired concentrations of insulin, transferrin, sodium selenite and ethanolamine were separately added according to a specific adaptation schedule. An exemplary, non-limiting, adaptation schedule is shown in Table 2 in the Examples.

In some embodiments, insulin and transferrin concentrations were gradually reduced during each passage until insulin and transferrin were no longer added to the culture media. In some embodiments, during the weaning process, if a split passage resulted in cultures in which the cell densities were lower than 1.8 million cells/mL, the cells were cultured in the media without reduction of insulin and transferrin. In some embodiments, when the cell densities increased to greater than 1.8 million cells/mL, the next step in insulin and transferrin reduction was performed.

In some embodiments, after eight passages of the gradual reduction, no additional insulin and transferrin were added to the culture media. In some embodiments, after the eighth passage, the cells were maintained with a spin passage using media, which does not contain insulin and transferrin, for adaptation for growth in growth media without exogenously provided insulin and transferrin.

### B. Media Composition

In some embodiments, the growth media contains one or more of amino acids, peptides, proteins, carbohydrates, essential metals, minerals, vitamins, buffering agents, anti-microbial agents, growth factors, and/or additional components.

### 1. Components

In some embodiments, the media contains one or more of the following components.

In some embodiments, the basal media contains amino acids.

In some embodiments, the basal media contains biotin.

In some embodiments, the basal media contains choline chloride.

In some embodiments, the basal media contains D-calcium pantothenate.

In some embodiments, the basal media contains folic acid.

In some embodiments, the basal media contains niacinamide.

In some embodiments, the basal media contains pyridoxine hydrochloride.

In some embodiments, the basal media contains riboflavin.

In some embodiments, the basal media contains thiamine hydrochloride.

In some embodiments, the basal media contains vitamin B12 (also known as cyanocobalamin).

In some embodiments, the basal media contains i-inositol (myo-inositol).

In some embodiments, the basal media contains calcium chloride.

In some embodiments, the basal media contains cupric sulfate.

In some embodiments, the basal media contains ferric nitrate.

In some embodiments, the basal media contains magnesium chloride.

In some embodiments, the basal media contains magnesium sulfate.

In some embodiments, the basal media contains potassium chloride.

In some embodiments, the basal media contains sodium bicarbonate.

In some embodiments, the basal media contains sodium chloride.

In some embodiments, the basal media contains sodium phosphate dibasic.

In some embodiments, the basal media contains sodium phosphate monobasic.

In some embodiments, the basal media contains zinc sulfate.

In some embodiments, the basal media contains linoleic acid.

In some embodiments, the basal media contains lipoic acid.

In some embodiments, the basal media contains putrescine-2HCl.

In some embodiments, the basal media contains 1,4 butanediamine.

In some embodiments, the basal media contains Pluronic F-68.

### 2. Sugars

In some embodiments, the growth medium contains sugars. In some embodiments, the sugars include but are not limited to D-glucose, galactose, fructose, mannose, or any combination thereof. In an embodiment, the sugars include both D-glucose and mannose.

In embodiments where glucose and mannose are both used in the growth medium to cultivate cells, the amount of glucose in the growth medium (cultivation media) is between 0.1-10 g/L, 0.1-9 g/L, 0.1-8 g/L, 0.1-7 g/L, 0.1-6 g/L, 0.1-5 g/L, 0.1-4 g/L, 0.1-3 g/L, 0.1-2 g/L, 0.1-1 g/L, 0.5-10 g/L, 0.5-9 g/L, 0.5-8 g/L, 0.5-7 g/L, 0.5-6 g/L, 0.5-5 g/L, 0.5-4 g/L, 0.5-3 g/L, 0.5-2 g/L, 0.5-1 g/L, 1-10 g/L, 1-9 g/L, 1-8 g/L, 1-9 g/L, 1-8 g/L, 1-7 g/L, 1-6 g/L, 1-5 g/L, 1-4 g/L, 1-3 g/L, 1-2 g/L, 2-10 g/L, 2-9 g/L, 2-8 g/L, 2-9 g/L, 2-8 g/L, 2-7 g/L, 2-6 g/L, 2-5 g/L, 2-4 g/L, 2-3 g/L, 3-10 g/L, 3-9 g/L, 3-8 g/L, 3-9 g/L, 3-8 g/L, 3-7 g/L, 3-6 g/L, 3-5 g/L, 3-4 g/L, 4-10 g/L, 4-9 g/L, 4-8 g/L, 4-9 g/L, 4-8 g/L, 4-7 g/L, 4-6 g/L, 4-5 g/L, 5-10 g/L, 5-9 g/L, 5-8 g/L, 5-9 g/L, 5-8 g/L, 5-7 g/L, or 5-6 g/L, and the amount of mannose in the growth media is between 0.1-10 g/L. 0.1-9 g/L, 0.1-8 g/L, 0.1-7 g/L, 0.1-6 g/L, 0.1-5 g/L, 0.1-4 g/L, 0.1-3 g/L, 0.1-2 g/L, 0.1-1 g/L, 0.5-10 g/L, 0.5-9 g/L, 0.5-8 g/L, 0.5-7 g/L, 0.5-6 g/L, 0.5-5 g/L, 0.5-4 g/L, 0.5-3 g/L, 0.5-2 g/L, 0.5-1 g/L, 1-10 g/L, 1-9 g/L, 1-8 g/L, 1-9 g/L, 1-8 g/L, 1-7 g/L, 1-6 g/L, 1-5 g/L, 1-4 g/L, 1-3 g/L, 1-2 g/L, 2-10 g/L, 2-9 g/L, 2-8 g/L, 2-9 g/L, 2-8 g/L, 2-7 g/L, 2-6 g/L, 2-5 g/L, 2-4 g/L, 2-3 g/L, 3-10 g/L, 3-9 g/L, 3-8 g/L, 3-9 g/L, 3-8 g/L, 3-7 g/L, 3-6 g/L, 3-5 g/L, 3-4 g/L, 4-10 g/L, 4-9 g/L, 4-8 g/L, 4-9 g/L, 4-8 g/L, 4-7 g/L, 4-6 g/L, 4-5 g/L, 5-10 g/L, 5-9 g/L, 5-8 g/L, 5-9 g/L, 5-8 g/L, 5-7 g/L, or 5-6 g/L. The skilled worker will understand that combinations of these amounts of glucose and mannose can be used, for example, between 2-5 grams of glucose and 1-4 grams of mannose.

### 3. Serum

In some embodiments, the media contains animal serum. In some embodiments, the media contains low amounts of animal serum or is animal serum free.

In some embodiments, the basal media contains fetal bovine serum.

In some embodiments, the serum is selected from bovine calf serum.

In some embodiments, the growth medium contains at least 10% fetal bovine serum. In certain embodiments, the cells are grown in a medium with at least 10% fetal bovine serum, followed by a reduction to less than 2% fetal bovine serum, followed by a reduction to less than 1% fetal bovine serum, or no fetal bovine serum, before recovering the cells.

In another embodiment, the culture media contains low serum including fetal bovine serum, fetal calf serum, or any animal derived serum. In certain embodiments, low serum contains less than 5%, 4.5%, 4.0%, 3.5%, 3.0%, 2.5%, 2.0%, 1.5%, 1.0%, 0.9%, 0.8%, 0.7%, 0.6%, 0.5%, 0.4%, 0.3%, 0.2%, 0.1%, or 0.05% bovine serum, fetal calf serum, or any animal derived serum before recovering the cells.

In certain embodiments, the serum (e.g., fetal bovine serum or fetal calf serum) is reduced to less than or equal to 1.9% serum before recovering the cells. In certain embodiments, the serum is reduced to less than or equal to 1.7% serum before recovering the cells. In certain embodiments, the serum is reduced to less than or equal to 1.5% serum before recovering the cells. In certain embodiments, the serum is reduced to less than or equal to 1.3% serum before recovering the cells. In certain embodiments, the serum is reduced to less than or equal to 1.1% serum before recovering the cells. In certain embodiments, the serum is reduced to less than or equal to 0.9% serum before recovering the cells. In certain embodiments, the serum is reduced to less than or equal to 0.7% serum before recovering the cells. In certain embodiments, the serum is reduced to less than or equal to 0.5% serum before recovering the cells. In certain embodiments, the serum is reduced to less than or equal to 0.3% serum before recovering the cells. In certain embodiments, the serum is reduced to less than or equal to 0.1% serum before recovering the cells. In certain embodiments, the serum is reduced to less than or equal to 0.05% serum before recovering the cells. In certain embodiments, the serum is reduced to about 0% serum before recovering the cells.

In another embodiment, the culture media contains no serum including fetal bovine serum, fetal calf serum, or any animal derived serum.

In some embodiments, the culture media is basal media. In some embodiments, the basal media is SKGM (Skeletal muscle cell growth media), DMEM (Dulbecco's Modified Eagle Medium), DMEM/F12, MEM (Minimum Essential Medium), HAMS's F10, HAM's F12, IMDM (Iscove's Modified Dulbecco's Medium), McCoy's Media and RPMI (Roswell Park Memorial Institute). These basal media are commercially available (for example, from ThermoFisher Scientific). In certain embodiments, the basal media is DMEM/F12 or IMDM/F12.

In some embodiments, the basal media is DMEM/F12 and is in a ratio of 3:1; 2:1; 1:1, 1:2, or 1:3. In certain embodiments, the basal media is DMEM/F12 and in a ratio of about 3:1, 2:1; 1:1, 1:2, or 1:3.

In some embodiments, the basal media is IMDM/F12 and is in a ratio of 3:1; 2:1; or 1:1, 1:2, or 1:3.

### C. Cell Proliferation

In some embodiments, the cell lines are immortalized. In some embodiments, the cell lines have high proliferation rates. In some embodiments, the cell lines are immortalized and have high proliferation rates. In some embodiments, the cell line(s) have a consistent doubling time and viable cell density when evaluated at greater than 200 passages.

In some embodiments, the cells are not recombinant or engineered (i.e., non-GMO). In some embodiments, the cells have not been exposed to any viruses and/or viral DNA. In certain embodiments, the cells are both not recombinant or have not been exposed to any viruses and/or viral DNA and/or RNA.

In some embodiments, proliferation is measured by any method known to one skilled in the art. In some embodiments, proliferation is measured through direct cell counts. In certain embodiments, proliferation is measured by a haemocytometer. In some embodiments, proliferation is measured by automated cell imaging. In certain embodiments, proliferation is measured by a Coulter counter.

In some embodiments, proliferation is measured by using viability stains. In certain embodiments, the stains used contain trypan blue.

In some embodiments, proliferation is measured by the total DNA. In some embodiments, proliferation is measured by Bromodeoxyuridine (BrdU) labelling. In some embodiments, proliferation is measured by metabolic measurements. In certain embodiments, proliferation is measured by using tetrazolium salts. In certain embodiments, proliferation is measured by ATP-coupled luminescence.

In some embodiments, one or more of the maintenance, proliferation, differentiation, lipid accumulation, lipid content, proneness to purification and/or harvest efficiency, growth rates, cell densities, cell weight, resistance to contamination, expression of endogenous genes and/or protein secretion, shear sensitivity, flavor, texture, color, odor, aroma, gustatory quality, nutritional quality, minimized growth-inhibitory byproduct secretion, and/or minimized media requirements, of cultivated animal cells, in any culture conditions, are improved by the use of one or more of growth factors, proteins, peptides, fatty acids, elements, small molecules, plant hydrolysates, directed evolution, genetic engineering, media composition, bioreactor design, and/or scaffold design. In certain embodiments, the fatty acids contain stearidonic acid (SDA). In certain embodiments, the fatty acids contain linoleic acid. In certain embodiments, the growth factor contains insulin or insulin like growth factor. In certain embodiments, the growth factor contains fibroblast growth factor or the like. In certain embodiments, the growth factor contains epidermal growth factor or the like. In certain embodiments, the protein contains transferrin. In certain embodiments, the element contains selenium. In certain embodiments, a small molecule contains ethanolamine. The amount of ethanolamine used in the cultivations is between 0.05-10 mg/L, 0.05-10 mg/L, 0.1-10 mg/L, 0.1-9.5 mg/L, 0.1-9 mg/L, 0.1-8.5 mg/L, 0.1-8.0 mg/L, 0.1-7.5 mg/L, 0.1-7.0 mg/L, 0.1-6.5 mg/L, 0.1-6.0 mg/L, 0.1-5.5 mg/L, 0.1-5.0 mg/L, 0.1-4.5 mg/L, 0.1-4.0 mg/L, 0.1-3.5 mg/L, 0.1-3.0 mg/L, 0.1-2.5 mg/L, 0.1-2.0 mg/L, 0.1-1.5 mg/L, and 0.1-1.0 mg/L. In certain embodiments, a small molecule contains a steroid or a corticosteroid. In certain embodiments, a small molecule contains dexamethasone. In certain embodiments, a small molecule contains ethanolamine. In certain embodiments, the growth medium contains blood proteins or plasma proteins. In certain embodiments blood protein is fetuin.

In certain embodiments, the media can be supplemented with plant hydrolysates. In certain embodiments, the hydrolysates contain yeast extract, wheat peptone, rice peptone, phytone peptone, yeastolate, pea peptone, soy peptone, pea peptone, potato peptone, mung bean protein hydrolysate, or sheftone. The amount of hydrolysate used in the cultivations is between 0.1 g/L to 5 g/L, between 0.1 g/L to 4.5 g/L, between 0.1 g/L to 4 g/L, between 0.1 g/L to 3.5 g/L, between 0.1 g/L to 3 g/L, between 0.1 g/L to 2.5 g/L, between 0.1 g/L to 2 g/L, between 0.1 g/L to 1.5 g/L, between 0.1 g/L to 1 g/L, or between 0.1 g/L to 0.5 g/L.

In some embodiments, a small molecule contains lactate dehydrogenase inhibitors. The production of lactate by the cells inhibits the growth of the cells. Exemplary lactate dehydrogenase inhibitors are selected from the group consisting of oxamate, galloflavin, gossypol, quinoline 3-sulfonamides, N-hydroxyindole-based inhibitors, and FX11 (CAS 213971-34-7). In some embodiments, the amount of lactate dehydrogenase inhibitor in the cultivation medium is between 1-500 mM, 1-400 mM, 1-300 mM, 1-250 mM, between 1-200 mM, 1-175 mM, 1-150 mM, 1-100 mM, 1-50 mM, 1-25 mM, 25-500 mM, 25-400 mM, 25-300 mM, 25-250 mM, 25-200 mM, 25-175 mM, 25-125M, 25-100 mM, 25-75 mM, 25-50 mM, 50-500 mM, 50-400 mM, 50-300 mM, 50-250 mM, 50-200 mM, 50-175 mM, 50-150 mM, 50-125 mM, 50-100 mM, 50-75 mM, 75-500 mM, 75-400 mM, 75-300 mM, 75-250 mM, 75-200 mM, 75-175 mM, 75-150 mM, 75-125 mM, 75-100 mM, 100-500 mM, 100-400 mM, 100-300 mM, 100-250 mM, 100-200 mM, 100-150 mM, 100-125 mM, and 100-500 mM.

In some embodiments, the cultivated animal cells are grown in a suspension culture system. In some embodiments, the cells are grown in a batch, fed-batch, semi continuous (fill and draw) or perfusion culture system or some combination thereof. When grown in suspension culture, the suspension culture can be performed in a vessel (cultivation tank, bioreactor)) of a desired size. The vessel is a size that is suitable for growth of cells without unacceptable rupture of the cells. In some embodiments, the suspension culture system can be performed in vessel that is at least 25 liters (L), 50 L, 100 L, 200 L, 250 L, 350 L, 500 L, 1000 L, 2,500 L, 5,000 L, 10,000 L, 25,000 L, 50,000 L, 100,000 L, 200,000 L, 250,000 L, or 500,000 L. For smaller suspension cultures, the cultivation of the cells can be performed in a flask that is least 125 mL, 250 mL, 500 mL, 1 L, 1.5 L, 2 L, 2.5 L, 3 L, 5 L, 10 L, or larger.

In some embodiments, the cell density of the suspension culture is between 0.25×10⁶ cells·ml, 0.5×10⁶ cells/ml and 1.0×10⁶ cells/ml, between 1.0×10⁶ cells/ml and 2.0×10⁶ cells/ml, between 2.0×10⁶ cells/ml and 3.0×10⁶ cells/ml, between 3.0×10⁶ cells/ml and 4.0×10⁶ cells/ml, between 4.0×10⁶ cells/ml and 5.0×10⁶ cells/ml, between 5.0×10⁶ cells/ml and 6.0×10⁶ cells/ml, between 6.0×10⁶ cells/ml and 7.0×10⁶ cells/ml, between 7.0×10⁶ cells/ml and 8.0×10⁶ cells/ml, between 8.0×10⁶ cells/ml and 9.0×10⁶ cells/ml, between 9.0×10⁶ cells/ml and 10×10⁶ cells/ml, between 10×10⁶ cells/ml and 15.0×10⁶ cells/ml, between 15×10⁶ cells/ml and 20×10⁶ cells/ml, between 20×10⁶ cells/ml and 25×10⁶ cells/ml, between 25×10⁶ cells/ml and 30×10⁶ cells/ml, between 30×10⁶ cells/ml and 35×10⁶ cells/ml, between 35×10⁶ cells/ml and 40×10⁶ cells/ml, between 40×10⁶ cells/ml and 45×10⁶ cells/ml, between 45×10⁶ cells/ml and 50×10⁶ cells/ml, between 50×10⁶ cells/ml and 55×10⁶ cells/ml, between 55×10⁶ cells/ml and 60×10⁶ cells/ml, between 60×10⁶ cells/ml and 65×10⁶ cells/ml, between 70×10⁶ cells/ml and 75×10⁶ cells/ml, between 75×10⁶ cells/ml and 80×10⁶ cells/ml, between 85×10⁶ cells/ml and 90×10⁶ cells/ml, between 90×10⁶ cells/ml and 95×10⁶ cells/ml, between 95×10⁶ cells/ml and 100×10⁶ cells/ml, between 100×10⁶ cells/ml and 125×10⁶ cells/ml, or between 125×10⁶ cells/ml and 150×10⁶ cells/ml.

In some embodiments, the cultivated animal cells are grown while embedded in scaffolds or attached to scaffolding materials. In some embodiments, the cultivated animal cells are differentiated or proliferated in a bioreactor and/or on a scaffold. In some embodiments, the scaffold contains at least one or more of a microcarrier, an organoid and/or vascularized culture, self-assembling co-culture, a monolayer, hydrogel scaffold, decellularized animal product, such as decellularized meat, decellularized connective tissue, decellularized skin, decellularized offal, or other decellularized animal byproducts, and/or an edible matrix. In some embodiments, the scaffold contains at least one of plastic and/or glass or other material. In some embodiments, the scaffold contains natural-based (biological) polymers chitin, alginate, chondroitin sulfate, carrageenan, gellan gum, hyaluronic acid, cellulose, collagen, gelatin, and/or elastin. In some embodiments, the scaffold contains an unmodified protein or a polypeptide, or a modified protein or modified polypeptide. The unmodified protein or polypeptide or modified protein or polypeptide contains proteins or polypeptides isolated from plants or other organisms. Exemplary plant protein isolates or plant protein concentrates contain pulse protein, vetch protein, grain protein, nut protein, macroalgal protein, microalgal protein, and other plant proteins. Pulse protein can be obtained from dry beans, lentils, mung beans, favabeans, dry peas, chickpeas, cowpeas, bambara beans, pigeon peas, lupins, vetches, adzuki, common beans, fenugreek, long beans, lima beans, runner beans, or tepary beans, soybeans, or mucuna beans. Vetch protein can be obtained from the genus Vicia. Grain protein can be obtained from wheat, rice, teff, oat, corn, barley, sorghum, rye, millet, triticale, amaranth, buckwheat, quinoa and other grains. Nut protein can be obtained from almond, cashew, pecan, peanut, walnut, macadamia, hazelnut, pistachio, brazil, chestnut, kola nut, sunflower seeds, pumpkin seeds, flax seeds, cacao, pine nut, ginkgo, and other nuts. Proteins obtained from animal source can also be used as scaffolds, including milk proteins, whey, casein, egg protein, and other animal proteins. In some embodiments, the self-assembling co-cultures contain spheroids and/or aggregates. In some embodiments, the monolayer is with or without an extracellular matrix. In some embodiments, the hydrogel scaffolds contain at least one of hyaluronic acid, alginate and/or polyethylene glycol. In some embodiments, the edible matrix contains decellularized plant tissue. Vegetable and animal protein, both modified or unmodified, can be extruded in an extrusion machine to prepare an extrudate that can be used as a scaffold for adherent cell culture of cultivated animal cells. Cultivated animal cells or cells isolated from the tissue of an animal, for example a cultivated Gallus Gallus cells or Bos taurus cells as disclosed herein can be processed through an extrusion machine to make an extrudate, which extrudate can be used as a scaffold for cultivation of Bos taurus cells.

In some embodiments, either primary or secondary Bos taurus cells are modified or grown as in any of the preceding paragraphs.

### D. Recovery of Cells

The cells can be recovered by any technique apparent to those of skill. In some embodiments the cells are separated from the growth media or are removed from a bioreactor or a scaffold. In certain embodiments, the cultivated animal cells are separated by centrifugation, a mechanical/filter press, filtration, flocculation or coagulation or gravity settling or drying or some combination thereof. In certain embodiments, the filtration method contains tangential flow filtration, vacuum filtration, rotary vacuum filtration and similar methods. In certain embodiments the drying can be accomplished by flash drying, bed drying, tray drying and/or fluidized bed drying and similar methods. In certain embodiments, the cells are separated enzymatically. In certain embodiments, the cells are separated mechanically.

### E. Cell Safety

In some embodiments, the population of cultivated animal cells is substantially pure.

In some embodiments, tests are administered at one or more steps of cell culturing to determine whether the cultivated animal cells are substantially pure.

In some embodiments, the cultivated animal cells are tested for the presence or absence of bacteria. In certain embodiments, the types of bacteria tested include, but are not limited to: Salmonella enteritidis, Staphylococcus aureus, Campylobacter jejunim, Listeria monocytogenes, Fecal streptococcus, Mycoplasma genus, Mycoplasma pulmonis, Coliforms, and Escherichia coli.

In some embodiments, components of the cell media, such as fetal bovine serum, are tested for the presence or absence of viruses. In certain embodiments, the viruses include, but are not limited to: Bluetongue, Bovine Adenovirus, Bovine Parvovirus, Bovine Respiratory Syncytial Virus, Bovine Viral Diarrhea Virus, Rabies, Reovirus, REV (reticuloendotheliosis virus), AEV (avian encephalomyelitis virus), ALV (avian leukosis virus) A, ALVB, ALVJ, FAV (fowl adenovirus) 1, FAV3, CAV (Chicken anemia virus), ARV (Avian Reovirus), Avian S. pullorum, Avian Mycoplasma Genus, Adeno-associated virus, BK virus, Epstein-Barr virus, Hepatitis A virus, Hepatitis B virus, Hepatitis C virus, Herpes Simplex 1, Herpes Simplex 2, Herpes virus type 6, Herpes virus type 7, Herpes virus type 8, HIV1, HIV-2, HPV-16, HPV 18, Human cytomegalovirus, Human Foamy virus, Human T-lymphotropic virus, John Cunningham virus, and Parvovirus B19.

In some embodiments, the tests are conducted for the presence or absence of yeast and/or molds.

In some embodiments, the tests are for metal concentrations by mass spectrometry, for example inductively coupled plasma mass spectrometry (ICP-MS). In certain embodiments, metals tested include, but are not limited to: arsenic, lead, mercury, cadmium, and chromium.

In some embodiments, the tests are for hormones produced in the culture. In certain embodiments, the hormones include, but are not limited: to 17β-estradiol, testosterone, progesterone, zeranol, melengesterol acetate, trenbolone acetate, megestrol acetate, chlormadinone acetate, dienestrol, diethylstilbestrol, hexestrol, taleranol, zearalanone, and zeranol.

In some embodiments, the tests are in keeping with the current good manufacturing process as detailed by the United States Food and Drug Administration.

### F. Phenotyping, Process Monitoring and Data Analysis

In some embodiments, the cells are monitored by any technique known to a person of skill in the art. In some embodiments, differentiation is measured and/or confirmed using transcriptional markers of differentiation after total RNA extraction using RT-qPCR and then comparing levels of transcribed genes of interest to reference, e.g., housekeeping genes.

### IV. Food Composition

In certain embodiments provided herein are food compositions or food products containing cultivated animal cells that are cultivated in vitro. In some embodiments, the cells are combined with other substances or ingredients to make a composition that is an edible food product composition. In certain embodiments, the cultivated animal cells are used alone to make a composition that is a food product composition. In certain embodiments, the food product composition is a product that resembles nuggets, tenders, bites, steak, roast, ground meat, hamburger patties, sausage, or feed stock.

In some embodiments, the recovered cultivated animal cells are prepared into a composition with other ingredients. In certain embodiments, the composition contains cell paste, mung bean, mung bean protein, fat, and/or water.

In certain embodiments, the food composition or food product has a wet cell paste content of at least 100%, 90%, 80%, 75%, 70%, 65%, 60%, 50%, 40%, 35%, 25%, 15%, 10%, 5% or 1% by weight. In certain embodiments, the food composition or food product has a wet cell paste content by weight of between 10%-20%, 20%-30%, 30%-40%, 40%-50%, 60%-70%, 80%-90%, or 90%-100%. In certain embodiments, the composition contains a pulse protein content by weight of at least 75%, 70%, 60%, 50%, 40%, 30%, 25%, 20%, or 15% by weight. In certain embodiments, the food composition or food product has a pulse protein content by weight of between 10%-20%, 20%-30%, 30%-40%, 40%-50%, 60%-70%, 80%-90%, or 90%-95%. In certain embodiments, the food composition or food product contains a fat content of at least 50%, 40%, 30%, 25%, 20%, 15%, 10%, 5%, or 1% by weight. In certain embodiments, the food composition or food product has a fat content by weight of between 10%-20%, 20%-30%, 30%-40%, 40%-50%, 60%-70%, 80%-90%, or 90%-95%. In certain embodiments, the food composition or food product contains a water content of at least 50%, 40%, 30%, 25%, 20%, 15%, 10% or 5% by weight. In certain embodiments, the food composition or food product has a water content by weight of between 10%-20%, 20%-30%, 30%-40%, 40%-50%, 60%-70%, 80%-90%, or 90-95%. In certain embodiments, the food composition or food product contains a wet cell paste content of between 2%-5%, 5%-10%, 10%-15%, 15%-20%, 20%-25%, 25%-30%, 30%-35%, 35%-40%, 40%-45%, 45%-50%, 50%-55%, 55%-60%, 65%-70%, 70%-75%, 75%-80%, 80%-85%, 85%-90%, or 90%-95%.

In some embodiments, the composition contains a peptide cross-linking enzyme, for example, transglutaminase, at a content from about to about, or between, 0.0001-0.0125%.

In certain embodiments, the food composition or food product contains a dry cell weight content of at least 1% by weight. In certain embodiments, the food composition or food product contains a dry cell weight content of at least 5% by weight. In certain embodiments, the food composition or food product contains a dry cell weight content of at least 10% by weight. In certain embodiments, the food composition or food product contains a dry cell weight content of at least 15% by weight. In certain embodiments, the food composition or food product contains a dry cell weight content of at least 20% by weight. In certain embodiments, the food composition or food product contains a dry cell weight content of at least 25% by weight. In certain embodiments, the composition or food product contains a dry cell weight of at least 30% by weight. In certain embodiments, the composition or food product contains a dry cell weight of at least 35% by weight. In certain embodiments, the composition or food product contains a dry cell weight of at least 40% by weight. In certain embodiments, the composition or food product contains a dry cell weight of at least 45% by weight. In certain embodiments, the composition or food product contains a dry cell weight of at least 50% by weight. In certain embodiments, the composition or food product contains a dry cell weight of at least 55% by weight. In certain embodiments, the composition or food product contains a dry cell weight of at least 60% by weight. In certain embodiments, the composition or food product contains a dry cell weight of at least 65% by weight. In certain embodiments, the composition or food product contains a dry cell weight of at least 70% by weight. In certain embodiments, the composition or food product contains a dry cell weight of at least 75% by weight. In certain embodiments, the composition or food product contains a dry cell weight of at least 80% by weight. In certain embodiments, the composition or food product contains a dry cell weight of at least 85% by weight. In certain embodiments, the composition or food product contains a dry cell weight of at least 90% by weight. In certain embodiments, the composition or food product contains a dry cell weight of at least 95% by weight. In certain embodiments, the composition or food product contains a dry cell weight of at least of 97% by weight. In certain embodiments, the composition or food product contains a dry cell weight of at least 98% by weight. In certain embodiments, the composition or food product contains a dry cell weight of at least 99% by weight. In certain embodiments, the composition or food product contains a dry cell weight of at least 100% by weight. In certain embodiments, the food composition or food product contains a dry cell weight content of between about 2%-5%, 5%-10%, 10%-15%, 15%-20%, 20%-25%, 25%-30%, 30%-35%, 35%-40%, 40%-45%, 45%-50%, 50%-55%, 55%-60%, 65%-70%, 70%-75%, 75%-80%, 80%-85%, 85%-90%, or 90%-95%,

In certain embodiments, the food composition or food product contains a pulse protein content of at least 2%, 5%, 10%, 15%, 20%, 25%, 30%, 35%, 40%, 45%, 50%, 55%, 60%, 65%, 70%, 75%, 80%, 85%, 90% or 95% by weight. In certain embodiments, the food composition or food product contains a pulse protein content of between 2%-5%, 5%-10%, 10%-15%, 15%-20%, 20%-25%, 25%-30%, 30%-35%, 35%-40%, 40%-45%, 45%-50%, 50%-55%, 55%-60%, 65%-70%, 70%-75%, 75%-80%, 80%-85%, 85%-90%, or 90%-95%, In some embodiments, the pulse protein is a mung bean protein.

In certain embodiments, the food composition or food product contains, a fat content of at least 1% by weight, a fat content of at least 2% by weight, a fat content of at least 5% by weight, a fat content of at least 7.5% by weight, or a fat content of at least 10% by weight. In certain embodiments, the food composition or food product contains a fat content of at least 15% by weight. In certain embodiments, the food composition or food product contains a fat content of at least 20% by weight. In certain embodiments, the food composition or food product contains a fat content of at least 25% by weight. In certain embodiments, the food composition or food product contains a fat content of at least 27% by weight. In certain embodiments, the food composition or food product contains a fat content of at least 30% by weight. In certain embodiments, the food composition or food product contains a fat content of at least 35% by weight. In certain embodiments, the food composition or food product contains a fat content of at least 40% by weight. In certain embodiments, the food composition or food product contains a fat content of at least 45% by weight. In certain embodiments, the food composition or food product contains a fat content of at least 50% by weight. In certain embodiments, the food composition or food product contains a fat content of at least 55% by weight. In certain embodiments, the food composition or food product contains a fat content of at least 60% by weight. In certain embodiments, the food composition or food product contains a fat content of at least 65% by weight. In certain embodiments, the food composition or food product contains a fat content of at least 70% by weight. In certain embodiments, the food composition or food product contains a fat content of at least 75% by weight. In certain embodiments, the food composition or food product contains a fat content of at least 80% by weight. In certain embodiments, the food composition or food product contains a fat content of at least 85% by weight. In certain embodiments, the food composition or food product contains a fat content of at least 90% by weight. In some embodiments, that food composition or food product contains a fat content of between 1%-5%, between 5%-10%, between 10%-15%, between 15%-20%, between 20%-25%, between 25%-30%, between 30%-35%, between 35%-40%, between 45%-50%, between 50%-55%, between 55%-60%, between 60%-65%, between 65%-70%, between 70%-75%, between 75%-80%, between 80%-85%, between 85%-90%, or between 90%-95%.

In certain embodiments, the food composition or food product contains a water content of at least 5% by weight. In certain embodiments, the food composition or food product contains a water content of at least 10% by weight. In certain embodiments, the food composition or food product contains a water to an amount of at least 15% by weight. In certain embodiments, the food composition or food product contains a water content of at least 20% by weight. In certain embodiments, the food composition or food product contains a water content of at least 25% by weight. In certain embodiments, the food composition or food product contains a water content of at least 30% by weight. In certain embodiments, the food composition or food product contains a water content of at least 35% by weight. In certain embodiments, the food composition or food product contains a water content of at least 40% by weight. In certain embodiments, the food composition or food product contains a water content of at least 45% by weight. In certain embodiments, the food composition or food product contains a water content to an amount of at least 50% by weight. In certain embodiments, the food composition or food product contains a water content to an amount of at least 55% by weight. In certain embodiments, the food composition or food product contains a water content to an amount of at least 60% by weight. In certain embodiments, the food composition or food product contains a water content to an amount of at least 65% by weight. In certain embodiments, the food composition or food product contains a water content to an amount of at least 70% by weight. In certain embodiments, the food composition or food product contains a water content to an amount of at least 75% by weight. In certain embodiments, the food composition or food product contains a water content to an amount of at least 80% by weight. In certain embodiments, the food composition or food product contains a water content to an amount of at least 85% by weight. In certain embodiments, the food composition or food product contains a water content to an amount of at least 90% by weight. In certain embodiments, the food composition or food product contains a water content to an amount of at least 95% by weight.

In one embodiment, the food composition or food product contains a wet cell paste content between 25-75% by weight, a mung bean protein content between 15-45% by weight, a fat content between 10-30% by weight, and a water content between 20-50% by weight.

In certain embodiments, the food composition or food product contains peptide cross-linking enzyme. Exemplary peptide cross-linking enzymes are selected from the group consisting of transglutaminase, sortase, subtilisin, tyrosinase, laccase, peroxidase, and lysyl oxidase. In certain embodiments, the composition contains a cross-linking enzyme of between 0.0001%-0.025%, 0.0001%-0.020%, 0.0001%-0.0175%, 0.0001%-0.0150%, 0.0001%-0.0125%, 0.0001%-0.01%, 0.0001%-0.0075%, 0.0001%-0.005%, 0.0001%-0.0025%, 0.0001%-0.002%, 0.0001%-0.0015%, 0.0001%-0.001%, 0.0001%-0.00015% by weight. In certain embodiments, the food composition or food product contains a transglutaminase content between 0.0001%-0.025%, 0.0001%-0.020%, 0.0001%-0.0175%, 0.0001%-0.0150%, 0.0001%-0.0125%, 0.0001%-0.01%, 0.0001%-0.0075%, 0.0001%-0.005%, 0.0001%-0.0025%, 0.0001%-0.002%, 0.0001%-0.0015%, 0.0001%-0.001%, 0.0001%-0.00015% by weight. Without being bound by theory, the peptide cross-linking enzyme is believed to cross-link the pulse or vetch proteins and the peptide cross-linking enzyme is believed to cross-link the pulse or vetch proteins to the Bos taurus cells.

In one embodiment, the food composition or food product contains 0.0001% to 0.0125% transglutaminase, and exhibits reduced or significantly reduced lipoxygenase activity or other enzymes which oxidize lipids, as expressed on a volumetric basis relative to cell paste without the transglutaminase. More preferably, the food composition or food product is essentially free of lipoxygenase or enzymes that can oxidize lipids. In some embodiments, a 5%, 10%, 15%, 20%, 25%, 30%, 35%, 40%, 45%, 50%, 55%, 60%, 65%, 70%, 75%, or 80% reduction in oxidative enzymatic activity relative to a composition is observed. Lipoxygenases catalyze the oxidation of lipids that contribute to the formation of compounds that impart undesirable flavors to compositions.

In some embodiments, mung bean protein is replaced by plant-based protein comprising protein from garbanzo, fava beans, yellow pea, sweet brown rice, rye, golden lentil, chana dal, soybean, adzuki, sorghum, sprouted green lentil, du pung style lentil, and/or white lima bean.

In some embodiments, the addition of additional edible ingredients can be used to prepare the food composition of food product. Edible food ingredients contain texture modifying ingredients such as starches, modified starches, gums and other hydrocolloids. Other food ingredients contain pH regulators, anti-caking agents, colors, emulsifiers, flavors, flavor enhancers, foaming agents, anti-foaming agents, humectants, sweeteners, and other edible ingredients.

In certain embodiments, the methods and food composition or food product contain an effective amount of an added preservative in combination with the food combination.

Preservatives prevent food spoilage from bacteria, molds, fungi, or yeast (antimicrobials); slow or prevent changes in color, flavor, or texture and delay rancidity (antioxidants); maintain freshness. In certain embodiments, the preservative is one or more of the following: ascorbic acid, citric acid, sodium benzoate, calcium propionate, sodium erythorbate, sodium nitrite, calcium sorbate, potassium sorbate, BHA (butylated hydroxyanisole), BHT (butylated hydroxytoluene), EDTA (Ethylenediaminetetraacetic acid), tocopherols (Vitamin E) and antioxidants, which prevent fats and oils and the foods containing them from becoming rancid or developing an off-flavor.

### V. Food Process

In some embodiments, provided herein are processes for making a food product that contains combining pulse protein, cultivated animal cell paste and a phosphate into water and heating up the mixture in three steps. In certain embodiments, the processes contain adding phosphate to water thereby conditioning the water to prepare conditioned water. In certain embodiments, pulse protein is added to the conditioned water in order to hydrate the pulse protein to prepare hydrated plant protein. In some embodiments, cell paste is added to the hydrated plant protein (conditioned water to which a plant protein has been added) to produce a cell protein mixture. In some embodiments, the plant protein is a pulse protein. In some embodiments, the pulse protein is a mung bean protein.

In some embodiments, the phosphate is selected from the group consisting of disodium phosphate (DSP), sodium hexametaphosphate (SHMP), tetrasodium pyrophosphate (TSPP). In one particular embodiment, the phosphate added to the water is DSP. In some embodiments, the amount of DSP added to the water is at least or about 0.01%, 0.02%, 0.03%, 0.04%, 0.05%, 0.06%, 0.07%, 0.08%, 0.09%, 0.1%, 0.11%, 0.12%, 0.13%, 0.14%, 0.15%, or greater than 0.15%.

In some embodiments, the process contains undergo three heating steps. In some embodiments, the first heating step contains heating the cell and protein mixture to a temperature between 40-65° C., wherein seasoning is added. In some embodiments, the second step contains maintaining the cell and protein mixture at temperature between 40-65° C. for at least 10 minutes, wherein a peptide cross-linking enzyme such as transglutaminase is added. In some embodiments, the third heating step contains raising the temperature of the cell and protein mixture to a temperature between 60-85° C., where oil is added to the water. In some embodiments, the process contains a fourth step of lowering the temperature to a temperature between 5-15° C. to prepare a pre-cooking product.

In some embodiments, the seasonings are added to the first step, second step, third step or the fourth step. In some embodiments the seasonings include but are not limited to salt, sugar, paprika, onion powder, garlic powder, black pepper, white pepper, and natural chicken flavor (Vegan).

In some embodiments, the oil (fat) added is to the first step, second step, third step or the fourth step to prepare the pre-cooking product. The oil is selected from the group comprising vegetable oil, peanut oil, canola oil, coconut oil, olive oil, corn oil, soybean oil, sunflower oil, margarine, vegetable shortening, animal oil, butter, tallow, lard, margarine, or an edible oil.

In some embodiments, the pre-cooking product can be consumed without additional preparation or cooking, or the pre-cooking product can be cooked further, using well-known cooking techniques.

In some embodiments, the food product is produced by other techniques known in the art including, but not limited to, extrusion. In some embodiments, the food product is formed by extruding a cell paste with a dry component (e.g., plant protein, flavoring agents, etc.). In some embodiments, a cell paste and dry component is extruded via an extruder having a cooling die that has a body defining a flow path extending from an inlet at an upstream end of the body to an outlet at a downstream end of the body. In some embodiments, the inlet has a first cross-sectional area, and the outlet has a second cross-sectional area that is larger than the first cross-sectional area. Additionally, in some embodiments, the flow path can include a transitional region at the upstream end of the body with a length L and a minimum cross-sectional expansion ratio of at least 2× for any continuous length L/2 over the length L of the transitional region, and the flow path includes a forming region extending from the transitional region to the outlet.

In some embodiments, the resulting extrudate has a texture similar to a farm-raised animal counterpart.

In some embodiments, the formulation can include cultivated animal cells in an amount between 35-95 wt % of the formulation, and a dry mix in an amount between 25-45 wt % of the formulation. The dry mix can include a proteinaceous ingredient, a binding ingredient, an emulsifier, and one or more flavorants.

In some embodiments, the extrudate includes an elongated body with a generally elliptical cross-section. In some embodiments, the elongated body includes irregular folds at a surface of the elongated body and disposed throughout a volume of the elongated body and the extrudate has a texture that is similar to a farm-raised animal counterpart.

Methods for forming an extrudate from a formulation including cultivated animal cells as described herein and a proteinaceous ingredient. In some embodiments, the method includes a step of receiving an intermediate extrudate into a flow path of a cooling die. In an exemplary embodiment, the flow path extends from an inlet with a first cross-sectional area at an upstream end of the cooling die to an outlet with a second cross-sectional area at a downstream end of the cooling die. Additionally, in some embodiments, the flow path includes a transition region at the upstream end and a forming region at the downstream end.

In some embodiments, the processes includes preparing the food product by placement into cooking molds. In some embodiments, the processes contain applying a vacuum to the cooking molds effectively changing the density and texture of the food product that contains cultivated animal cells.

In some embodiments, the food product is breaded.

In some embodiments, the food product is steamed, boiled, sautéed, fried, baked, grilled, broiled, microwaved, dehydrated, cooked by sous vide, pressure cooked, or frozen or any combination thereof.

### EXAMPLES

All the provided Examples provide additional description of disclosed embodiments, and thus all elements, compositions, methods steps, etc. of the Examples and their associated experiments are expressly provided individual and in any and all possible combinations with any and all other elements, compositions, and method steps, etc. provided elsewhere herein.

### Example 1: Avian Cells

The weaning of insulin and transferrin was performed in cultivated chicken cells. The weaned cells had no compromise in cell growth and the growth parameters were comparable to cells requiring insulin and transferrin in the growth media.

Initially, concentrations of insulin and transferrin were gradually reduced from culture media. Once insulin and transferrin were completely removed from growth media, cell adaptation was initiated by using the growth media without the addition of insulin and transferrin by spin passage. When cell growth was stabilized with consistent cell growth, research cell banks were generated.

Two vials of frozen chicken cells (C1F-P2) that required insulin and transferrin for cell growth were thawed into a suspension condition in the presence of insulin and transferrin with a working volume of 50 mL in a 125 mL shake flask. Cells were cultured in a shaking incubator at 37° C., 5% CO₂ in a humidified (70-80%) incubator. At the first passage immediately after thawing, cells were spin passaged in the presence of insulin and transferrin with a working volume of 50 mL in a 125 mL shake flask. The cells were passaged a second time in the presence of insulin and transferrin and the cells were split passaged for a third time in the presence of insulin and transferrin at a target inoculation density of 0.5 million cells/mL. After the third passage, cells were maintained in culture media containing insulin and transferrin with 1:3 split passages until they were used to wean the cells from insulin and transferrin. Culture volume was scaled up during cell maintenance to 900 mL.

Chemically defined serum free media was used for cell maintenance and adaptation. Composition of the culture media is described in Table 1. For cell maintenance, cells were cultured in media containing DMEM/F12, sodium bicarbonate, glucose, glutamine, sodium chloride, poloxamer, biotin, folic acid, vitamin 12, niacin, ITSX, and lipids. Osmolality of culture media was 280 mOsm/Kg and pH was 7.6

TABLE 1
Culture Media Components

| | | SKU/ |
|---|---|---|
| Component | Vendor | Catalog # |
| DMEM/F12 | ThermoFisher | A42914EK |
| Glucose | Spectrum Chemical | D1017 |
| Glutamine | Gibco | 21051-040 |
| Sodium chloride | Spectrum Chemical | S0160 |
| Poloxamer 188 | Spectrum Chemical | P1169 |
| Sodium Bicarbonate | Sigma | S5761 |
| Vitamin B12 | Alfa Aesar | A14894-06 |
| Niacin | Spectrum | NI00-500GM |
| Biotin | Alfa Aesar | A14207-03 |
| Folic acid | Spectrum Chemical | F0I05-25GM |
| ITS-X (100X) | Gibco | ME20396L1 |
| CD lipids (CDL) | ThermoFisher | 11905 |
| (1000X) | | |
| Trace A (1000X) | Corning | 25-025 |
| RO/DI Water | Internal | N/A |

For cell adaptation to insulin free and transferrin free culture media, ITSX (a commercially available reagent that includes insulin, transferrin, sodium selenite and ethanolamine) was removed from the culture media. Desired concentrations of insulin, transferrin, sodium selenite and ethanolamine were separately added according to the adaptation schedule as shown in Table 2.

TABLE 2
Insulin and Transferrin Weaning Schedule

| | Insulin | Transferrin | |
|---|---|---|---|
| | conc., | conc., | |
| Step | mg/L | mg/L | Notes |
| 1 | 10 | 5 | Insulin and transferrin concentrations in |
| | | | maintenance media (full insulin and |
| | | | transferrin) |
| 2 | 8 | 4 | 20% reduction of insulin and transferrin |
| 2 | 8 | 4 | No insulin and transferrin reduction |
| 3 | 6 | 3 | 40% reduction of insulin and transferrin |
| 4 | 4 | 2 | 60% reduction of insulin and transferrin |
| 5 | 2 | 1 | 80% reduction of insulin and transferrin |
| 6 | 1 | 0.5 | 90% reduction of insulin and transferrin |
| 7 | 1 | 0.5 | 95% reduction of insulin and transferrin |
| 8 | 0.5 | 0.25 | No insulin and transferrin reduction |
| 8 | 0 | 0 | No exogenously provided insulin and |
| | | | transferrin |

Table 3 shows the concentrations, source and catalog numbers of insulin, transferrin, sodium selenite and ethanolamine of the stock solutions. To dissolve 10 mg insulin in 10 mL DI water, 10 μL of 6N hydrochloric acid was added. Insulin and transferrin stock solutions were prepared and stored at −20° C. until needed. Once insulin and transferrin stock solutions were thawed, desired amounts of insulin and transferrin stock solutions were used to prepare growth media with the appropriate reductions in insulin and transferrin as shown in Table 2. All leftover unused aliquots of insulin and transferrin were discarded. Sodium Selenite and Ethanolamine stock solutions were prepared prior to the preparation of each media batch.

TABLE 3
Sources of ITSX

| | | | Final concentrations | |
|---|---|---|---|---|
| | | SKU/ | in usual culture | |
| | Vendor | Catalog # | media | |
| Insulin | Gibco | 12605-010 | 10 | mg/L |
| Transferrin | InVitria | 777TRF029-1G | 5.5 | mg/L |
| Sodium Selenite | Sigma | S5261-25G | 0.0067 | mg/L |
| Ethanolamine | Acros | 141-43-5 | 2 | mg/L |

Cell maintenance and subcultures were carried out in maintenance culture media as shown in Table 1 with 1:3 split passage. In the 1:3 split passage method, one third of the total culture volume was transferred into the shake flasks containing two thirds of the total culture volume of fresh media.

Cell counts were performed using ViCell XR Cell Viability Analyzer (Beckman Counter) for the quantification of cell density and viability. A total of 0.8 mL was taken from the culture into 1.5 mL microtubes. Samples were centrifuged at 300×g for 5 min, and the supernatant was removed. Cell pellets were resuspended with a 300 μL of TrypLE Express (Gibco) and incubated at 37° C. for 5 min inside a shaking incubator. After incubation, 300 μL of culture media was added into the cells to dilute the enzymatic activity of TrypLE. The 600 μL total volume was immediately processed by the ViCell XR to measure cell density, viability, and viable cell density.

Based on cell counting results, population doubling time (PDT) and Population doubling level (PDL) were calculated according to the following formulas:

PDT=t*log 10(2)/((log 10(n/n0)), where t=culture time,n=final cell number and n0=number of cells seeded.

PDL=3.32[log 10(n/n0)], where n=final cell number and n0=number of cells seeded

To expand cells for creation of a Research Cell Bank (RCB), culture volume was scaled up to 250 mL in 500 mL shake flasks initially, and then increased to 800 mL in 2.8 L shake flasks. Next, research cell banks (RCB) were prepared from actively growing cells. The volume of cell suspension that held the number of cells desired to bank was centrifuged at 500×g for 10 min, and the supernatant was removed. The cell pellets were gently resuspended with cryopreservation media. Total volume of cryopreservation media was decided to make the final cell density of 10 million cells/mL. A total of 15 million cells was aliquoted into each cryovial. Cryovials were stored in isopropanol chambers to freeze at a rate of −1° C./min from 4° C. to −80° C. After the freezing period of 24 to 72 hr in isopropanol chambers at −80° C., cells were transferred and stored in a vapor phase liquid nitrogen storage system.

Cell adaptation to the media without exogenously provided insulin and transferrin was performed with a spin passage at a targeted inoculation density of ˜0.5 million cells/mL. Prior to spin passage, aliquots of 100 mL fresh media were prepared into 250 mL shake flasks and prewarmed inside a shaking incubator. Based on the cell counts on harvest day, culture volumes were taken into 50 mL conical tubes. Cells were spun down at 450×g for 5 min and the supernatant was discarded. Cell pellets were gently resuspended with a portion of fresh media which was prewarmed prior to spin passage. Once cell pellets were completely dissociated, cells were inoculated.

In eight passages after thawing, insulin and transferrin concentrations were gradually reduced to no addition of exogenously provided insulin and transferrin in the growth media. Gradual reduction of insulin and transferrin was performed with a spin passage to completely remove residual insulin and/or transferrin from the previous passage. The schedule of insulin and transferrin reduction is shown in Table 2.

Insulin and transferrin concentrations were gradually reduced during each passage until insulin and transferrin were not added to the culture media. During the weaning process, if a split passage resulted in cultures in which the cell densities were lower than 1.8 million cells/mL, the cells were cultured in the media without reduction of insulin and transferrin. When the cell densities increased to greater than 1.8 million cells/mL, the next step in insulin and transferrin reduction was performed.

After eight passages of the gradual reduction, no additional insulin and transferrin were added to the culture media. After the eighth passage, the cells were maintained with a spin passage using media, which does not contain insulin and transferrin for adaptation for growth in growth media without exogenously provided insulin and transferrin.

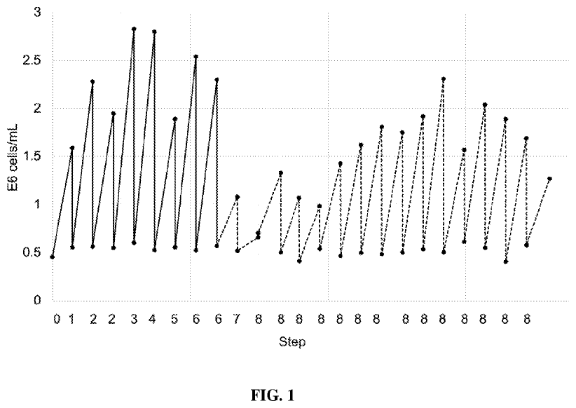

Initially, cell growth was significantly depressed during the early stages of adaptation. The dotted line in FIG. 1 shows viable cell density (VCD) during the adaptation for growth in media without insulin and transferrin. Specifically, at the second passage step, cells barely grew, obtaining a VCD of ˜0.7 million cells/ml and population doubling time (PDT) of ˜210 hr. However, after six passages of adaptation, cell proliferation improved and consistent cell growth was obtained with VCD of 1.5 to 2 million cells/mL and PDT of tens of hours, which were slightly lower than the usual C1F-P2 growth (VCD of ˜2 million cells/mL) parameters.

RCB was generated using cells adapted to the media without insulin and transferrin. To expand cells for RCB generation, culture volume was scaled up to 800 mL after 14 passages of adaptation. During the scale-up of culture, cell growth was maintained consistently with viabilities of >90%.

The adapted cells (C1F-P3) consistently reached VCD of 1.5 to 2 million cells/mL with a PDT of tens of hours.

### Example 2: Bovine Cells

One vial of B1E-S2 cells, adapted as described in Example 7, was thawed into suspension culture with a working volume of 30 mL in a 125 mL shake flask. Cells were cultured in a shaking incubator receiving 125 rpm shaking at 37° C., 5% CO₂ in a humidified (70-80%) atmosphere. The initial passage from thaw and each subsequent passage received spin passaging with a target inoculation density of 0.5 million cells/mL. Cells were maintained for three passages with insulin and transferrin prior to beginning the weaning protocol. Cells were maintained for three passages with insulin and transferrin prior to beginning the weaning protocol.

Chemically defined serum-free growth medium was used for cell maintenance and adaptation. This formula contains DMEM/F12, sodium bicarbonate, glucose, glutamine, sodium chloride, polaxamer, biotin, folic acid, vitamin B12, niacin, ITSX, and lipids. Osmolality of the culture media was 280 mOsm/kg and pH was 7.6. See Table 1 in Example 1.

For insulin and transferrin weaning, ITSX was removed from the culture media formulation. Appropriate concentrations of insulin, transferrin, sodium selenite, and ethanolamine were added separately according to the adaptation schedule of Table 4. Table 3 in Example 1 shows the stock concentrations of each stock solution.

Cell adaptation to media with reduced insulin and transferrin was conducted with spin passaging to remove the previous media condition and resuspension in fresh media with the appropriate concentration of insulin and transferrin, targeting an inoculation density of 0.5 million cells/mL. Prior to spin passaging, 100 mL of fresh media without ITSX was aliquot into 250 mL shake flasks and prewarmed inside of a shaking, 37° C. incubator. A volume containing 50 million cells was determined by cell counts at the time of harvest and collected into a 50 mL conical tube. These cells were centrifuged at 400×g for 5 minutes and the supernatant was discarded. The cell pellet was resuspended with a portion of the prewarmed, fresh media and inoculated into the 250 mL shake flask. Freshly prepared sodium selenite and ethanolamine were added to the inoculum. Insulin and transferrin were then added to the inoculum in appropriate concentrations as shown in Table 4. Cell counts were performed as described in Example 1.

TABLE 4
Insulin and Transferrin Weaning Schedule

| | Insulin | Transferrin | |
|---|---|---|---|
| | conc. | conc. | |
| Step | (mg/L) | (mg/L) | Notes |
| 1 | 10 | 5 | Insulin and transferrin concentrations in |
| | | | maintenance media (full insulin and transferrin |
| 2 | 6 | 3 | 40% reduction of insulin and transferrin |
| 3 | 4 | 2 | 60% reduction of insulin and transferrin |
| 3 | 4 | 2 | No insulin and transferrin reduction |
| 4 | 2 | 1 | 80% reduction of insulin and transferrin |
| 5 | 1 | 0.5 | 90% reduction of insulin and transferrin |
| 5 | 1 | 0.5 | No insulin and transferrin reductions |
| 6 | 0 | 0 | No exogenously provided insulin and transferrin |
| 6 | 0 | 0 | No exogenously provided insulin and transferrin |
| 6 | 0 | 0 | No exogenously provided insulin and transferrin |

To preserve the adaptation status at 100%-reduced concentrations of insulin and transferrin, an RCB was generated from step 10 cells. The volume of cell suspension that contained the desired number of cells for banking was transferred to 50 mL conical tubes and centrifuged at 400×g for 5 minutes. The cell pellets were resuspended with cryopreservation solution that had been pre-chilled to 4° C. The volume of cryopreservation solution used achieved a final cell density of 10 million cells/mL. A total of 15 million cells were aliquot into each cryovial. Cryovials were stored in isopropanol chambers to freeze at a rate of −1° C./minute from 4° C. to −80° C. After a 24-hour freezing period in −80° C., the RCB was transferred and stored in a vapor phase liquid nitrogen storage system.

B1E-S2 cells were initiated from thaw and subsequently cultured for 3 passages in serum-free medium containing 100% insulin and transferrin before weaning the cells from insulin and transferrin. After an initial recovery passage, B1E-S2 cells consistently grew to over 2 million cells/mL from a starting density of 0.5 million cells/mL, with cell viability >90%. Following 3 passages from thaw in complete ITSX, the concentrations of insulin and transferrin were gradually reduced according to the protocol in Table 4.

Cell viable cell density (VCD) dropped slightly to about 2.0 million cell/mL upon insulin and transferrin reduction from 40% to 60%, though viabilities remained above 90%. Cells were kept in the 60%-reduction condition for an additional passage instead of reducing the concentration further. At the next passage, VCD was consistent with the previous passage and viability was stable, so gradual reductions were continued towards complete removal of insulin and transferrin. The cells were maintained in insulin- and transferrin-free conditions for three consecutive passages, receiving complete media exchange at each passage.

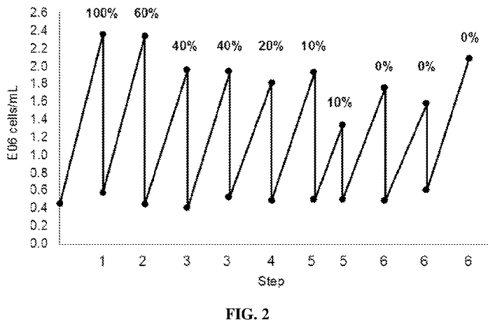

Within 6 passages, insulin and transferrin were weaned from 100% to 0%; FIG. 2 (dashed line) shows that cell growth was not substantially compromised from gradual reduction, and ultimate removal, of insulin and transferrin from culture. Cell growth during reduction was comparable to that measured prior to reduction, with comparable doubling times (PDT) during and after complete insulin and transferrin weaning. Viabilities remained stable above 85% over the course of weaning. FIG. 2 shows the VCD values of the B1E cells during and after the weaning process. The solid line shows the VCD of the cells grown in 100% insulin and transferrin. The dashed line shows the VCD of the cells grown in 10% insulin and transferrin.

Bovine master cell banks (MCB) were prepared by expanding the insulin and transferrin weaned bovine RCB cells.

### Example 3: Testing Safety of Bovine Cells for Bacteria and Viruses

Safety and efficacy of the cells is checked at all stages of growth and harvesting of the cells. Cultured Bos taurus cells are evaluated for presence of viral, yeast, and bacterial adventitious agents.

The cells are analyzed for the presence of bacteria using protocols from the FDA's Bacteriological Analytical Manual (BAM).

Total Plate Count (TPC) is synonymous with Aerobic Plate Count (APC). As indicated in the US FDA's BAM, Chapter 3, the assay is intended to indicate the level of microorganism in a product. Briefly, the method involves appropriate decimal dilutions of the sample and plating onto non-selective media in agar plates. After incubating for approximately 48 hours, the colony forming units (CFUs) are counted and reported as total plate count.

Yeast and mold are analyzed according to methodology outlined in the US FDA BAM, Chapter 18. Briefly, the method involves serial dilutions of the sample in 0.1% peptone water and dispensing onto a petri plate that contains nutrients with antibiotics that inhibit microbial growth but facilitate yeast and mold enumeration. Plates are incubated at 25° C. and counted after 5 days. Alternately, yeast and mold are analyzed by using ten-fold serial dilutions of the sample in 0.1% peptone water and dispensing 1 mL onto Petrifilm that contains nutrients with antibiotics that facilitate yeast and mold enumeration. The Petrifilm is incubated for 48 hours incubated at 25 or 28° C. and the results are reported as CFUs.

Escherichia coli and coliform are analyzed according to methodology outlined in the US FDA BAM, Chapter 4. The method involves serial decimal dilutions in lauryl sulfate tryptone broth and incubated at 35° C. and checked for gas formation. Next step involves the transfer from gassing tubes (using a 3 mm loop) into BGLB broth and incubated at 35° C. for 48+/−2 hours. The results are reported as MPN (most probable number) coliform bacteria/g.

Streptococcus is analyzed using CMMEF method as described in chapter 9 of BAM. The assay principle is based on the detection of acid formation by Streptococcus and indicated by a color change from purple to yellow. KF Streptococcus agar medium is used with triphenyl tetrazolium chloride (TTC) for selective isolation and enumeration. The culture response is reported as CFUs after incubating aerobically at 35+/−2° C. for 46-48 hours.

Salmonella is analyzed according to methodology outlined in the US FDA BAM, Chapter 5. Briefly, the analyte is prepared for isolation of Salmonella then isolated by transferring to selective enrichment media, the plated onto bismuth sulfite (BS) agar, xylose lysine deoxycholate (XLD) agar, and Hektoen enteric (HE) agar. This step is repeated with transfer onto RV medium. Plates are incubated at 35° C. for 24+/−2 hours and examined for presence of colonies that may be Salmonella. Presumptive Salmonella are further tested through various methodology to observe biochemical and serological reactions of Salmonella according to the test/substrate used and result yielded.

Cultured Bos taurus cells are considered acceptable for Mycoplasma for example, if a minimum 3% of randomly selected and tested cell vials from each bank are thawed and their culture supernatants provide a negative result using the MycoAlert™ Mycoplasma Detection Kit. Following the kit guidelines, the tested samples are classified according to the ratio between Luminescence Reading B and Luminescence Reading A: Ratio <0.9 Negative for Mycoplasma; 0.9<Ratio<1.2 Borderline (required retesting of cells after 24 hours); Ratio>1.2 Mycoplasma contamination.

Viral assessment can be performed by analyzing adventitious human virus and bacterial agents through an Infectious Disease Polymerase Chain Reaction (PCR) performed in-house or by a third-party (Charles River Research Animal Diagnostic Services)—Human Essential CLEAR Panel; Bacteria Panel.

Bos taurus cell banks are considered valid for viral assessment if a minimum of 3% of independent cell vials from the tested bank are thawed and their cell pellets provide a negative result for the full panel of adventitious agents.

Cultured Bos taurus cells are considered approved for absence of adventitious human viral and bacterial agents if the independent cell pellets from each cell bank are negative for the entire human panels.

Detection of adventitious contaminations are performed by testing bovine cells. Bovine cells are considered valid for viral assessment if a minimum of 0.4×\sqrt{n} of randomly selected and tested cryovials from each bank of cells (of “n” bank size) are thawed and their cell pellets provide a negative result for the full panel of adventitious agents listed in Table 5.

TABLE 5
Panel of human adventitious agents tested in bovine cells.

Adeno-associated virus
BK virus
Epstein-Barr virus
Hepatitis A virus
Hepatitis B virus
Hepatitis C virus
Herpes Simplex 1 PCR
Herpes Simplex 2 PCR
Herpesvirus type 6
Herpesvirus type 7
Herpesvirus typc 8
HIV-1
HIV-2
HPV-16
HPV-18
Human cytomegalovirus
Human Foamy virus
Human T-lymphotropic virus
John Cunningham virus
Parvovirus B19
Mycoplasma Genus PCR
Mycoplasma pulmonis PCR

### Example 4: Testing Safety of Avian Cells for Bacteria and Viruses

Safety and efficacy of the cells is checked at all stages of growth and harvesting of the cells. Cultured C1F cells are evaluated for presence of viral, yeast, and bacterial adventitious agents.

The chicken product is analyzed for the presence of bacteria using protocols from the FDA's Bacteriological Analytical Manual (BAM).

Total Plate Count (TPC) is synonymous with Aerobic Plate Count (APC). As indicated in the US FDA's BAM, Chapter 3, the assay is intended to indicate the level of microorganism in a product. Briefly, the method involves appropriate decimal dilutions of the sample and plating onto non-selective media in agar plates. After incubating for approximately 48 hours, the colony forming units (CFUs) are counted and reported as total plate count.

Yeast and mold are analyzed according to methodology outlined in the US FDA BAM, Chapter 18. Briefly, the method involves serial dilutions of the sample in 0.1% peptone water and dispensing onto a petri plate that contains nutrients with antibiotics that inhibit microbial growth but facilitate yeast and mold enumeration. Plates are incubated at 25° C. and counted after 5 days. Alternately, yeast and mold are analyzed by using ten-fold serial dilutions of the sample in 0.1% peptone water and dispensing 1 mL onto Petrifilm that contains nutrients with antibiotics that facilitate yeast and mold enumeration. The Petrifilm is incubated for 48 hours incubated at 25 or 28° C. and the results are reported as CFUs.

Escherichia coli and coliform are analyzed according to methodology outlined in the US FDA BAM, Chapter 4. The method involves serial decimal dilutions in lauryl sulfate tryptone broth and incubated at 35° C. and checked for gas formation. Next step involves the transfer from gassing tubes (using a 3 mm loop) into BGLB broth and incubated at 35° C. for 48+/−2 hours. The results are reported as MPN (most probable number) coliform bacteria/g.

Streptococcus is analyzed using CMMEF method as described in chapter 9 of BAM. The assay principle is based on the detection of acid formation by Streptococcus and indicated by a color change from purple to yellow. KF Streptococcus agar medium is used with triphenyl tetrazolium chloride (TTC) for selective isolation and enumeration. The culture response is reported as CFUs after incubating aerobically at 35+/−2° C. for 46-48 hours.

Salmonella is analyzed according to methodology outlined in the US FDA BAM, Chapter 5. Briefly, the analyte is prepared for isolation of Salmonella then isolated by transferring to selective enrichment media, the plated onto bismuth sulfite (BS) agar, xylose lysine deoxycholate (XLD) agar, and Hektoen enteric (HE) agar. This step is repeated with transfer onto RV medium. Plates are incubated at 35° C. for 24+/−2 hours and examined for presence of colonies that may be Salmonella. Presumptive Salmonella are further tested through various methodology to observe biochemical and serological reactions of Salmonella according to the test/substrate used and result yielded. Quantities tested from 500 L harvests will be consistent with FDA BAM-Chapter 5.

Cultured chicken was prepared by methods consistent with the examples above. Table 1 indicates that bacteria contamination was negligible when compared to US FDA guidelines.

TABLE 6
Microbiological analysis of Cultured Chicken Meat

| | Basis | | | Representative | |
|---|---|---|---|---|---|
| Parameter | Method | Specification | | example | |
| Aerobic plate count | FDA BAM-Chapter 3 | <10,000 | cfu/g | <10 | cfu/g |
| Coliforms | FDA BAM-Chapter 4 | <3 | MPN/g | <3 | MPN/g |
| E. Coli | FDA BAM-Chapter 4 | <3 | MPN/g | <3 | MPN/g |
| Fetal Streptococcus | CMMEF-Chapter 9 | <10 | cfu/g | <10 | cfu/g |

| | | | |
|---|---|---|---|
| Salmonella | FDA BAM-Chapter 9 | Note Detected | Not Detected |

### Mycoplasma Contamination

Cultured C1F cells are considered valid for Mycoplasma detection if a minimum 3% of randomly selected and tested cell vials from each bank are thawed and their culture supernatants provide a negative result using the MycoAlert™ Mycoplasma Detection Kit. Following the kit guidelines, the tested samples are classified according to the ratio between Luminescence Reading B and Luminescence Reading A: Ratio <0.9 Negative for Mycoplasma; 0.9<Ratio<1.2 Borderline (required retesting of cells after 24 hours); Ratio>1.2 Mycoplasma contamination.

### Viral Assessment

Viral assessment was performed by analyzing adventitious human and avian virus and bacterial agents through an Infectious Disease Polymerase Chain Reaction (PCR) performed by a third-party (Charles River Research Animal Diagnostic Services)—Human Essential CLEAR Panel; Avian Virus and Bacteria Panel.

C1F from cell banks are considered valid for viral assessment if a minimum of 3% of independent cell vials from the tested bank are thawed and their cell pellets provide a negative result for the full panel of adventitious agents.

Cultured C1F cells are considered approved for absence of adventitious avian and human viral and bacterial agents as the independent cell pellets from each cell bank were negative for the entire human and avian panels.

### Example 5: Animal Cell Food Product Composition

A representative food product composition is described below (by weight percentage) in Table 7.

TABLE 7
Example bovine food product composition.

| | % |
|---|---|
| | by |
| Ingredient | weight |
| Water | 20-40 |
| Cell paste | 25-90 |
| Mung bean | 5-20 |
| Fat | 5-20 |
| transglutaminase | 0.000-0.05 |

The embodiments and examples described above are intended to be merely illustrative and non-limiting. Those skilled in the art will recognize or will be able to ascertain using no more than routine experimentation, numerous equivalents of specific compounds, materials and procedures. All such equivalents are considered to be within the scope and are encompassed by the appended claims.

### Example 6: Bioreactor Batch Cell Cultivation

The CIF-P2 and C1F-P3 cells of Example 1 were cultivated as suspension cultures in ambr250 bioreactors using the growth conditions and culture of Example 1. The osmolality of the cultivations was maintained between 275-300 mOsm/kg across different seed train shake flasks and production ambr250 bioreactors.

The cells were maintained in culture in suspension and expanded in shake flasks with or without insulin and transferrin by passaging every 3 days with seeding density at 0.5×10⁶ cells/mL.

The seed train was carried out in a 125 mL shake flask (Thomson) from cell thaw with a working volume of 50-60 mL and kept in an orbital shaker incubator (Infors HT, Annapolis Junction, MD, USA), with agitation at 125 rpm maintained at 37° C., 5% CO₂ and 80% humidified atmosphere. Seed train shake flasks were scaled up to 2.7 L working volume before inoculation in ambr250 production bioreactors. 50-60 mL of a seed train run was expanded to 2.7 L working volume for use as an inoculum into 4×200 mL ambr250 bioreactor for production of cultivated cells.

Batch cell cultures were started in duplicates using ambr250 bioreactor (Sartorius) at 200 mL working volumes with a 1:3 split passage from inoculum. Bioreactors were inoculated at seeding densities of 0.5-0.8×10⁶ cells/mL. All bioreactor cell cultures were maintained at 37° C., pH 7.4, 50% air saturation, and agitation speed of 350 rpm. Cell culture samples were taken from each bioreactor every 24 h for the determination of viable cell concentration, viability and other bioprocess parameters for process analyses. After every 3 days of cell growth, cell culture from each bioreactor were passaged (drain/refill) either with 1:3 or 1:4 split using a fresh media. The production run lasted for a duration of about 15 days. Table 8 below shows the experimental design for the production run using the ambr250 bioreactor.

TABLE 8
Batch Production Design

| | | | Media |
|---|---|---|---|
| | | Media | type |
| | | type | in |
| Bioreactor | Cell | in | production |
| ID | line | seed-train | bioreactor |
| 1 & 2 (Positive | C1F-P2L2 | Regular (+insulin/transferrin) | Regular (+insulin/transferrin) |
| control) | | | |
| 3 & 4 (Negative | C1F-P2L2 | Regular (+insulin/transferrin) | No insulin & transferrin |
| control) | | | |
| 5 & 6 | C1F-P3 | No insulin & transferrin | No insulin & transferrin |

The viabile cell densities (VCDs) of both C1F-P2L2 and C1F-P3 seed train cell cultures all reached up to 2×10⁶ cells/mL. The VCDs of the final seed runs used to inoculate the ambr250 were about 2×10⁶ cells/mL. C1F-P2L2 cells are a clonal cell line of C1F-P2 cells.

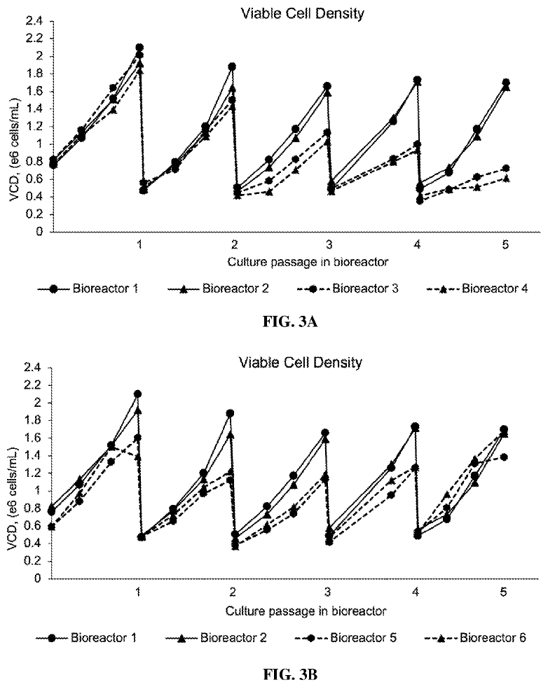

The bioreactors were inoculated with the seed train on day 0 and were passaged every three days. Bioreactors 1 and 2 were passaged with 1:3 split, and Bioreactors 3-6 were passaged with a 1:4 split. FIG. 3 shows the VCDs of bioreactors 1-6 during 360 hours of cultivation during which 4 harvests of cells were carried out.

Bioreactors 1 and 2, C1F-P2L2 cells cultivated with exogenously provided insulin and transferrin, reached VCDs of between 1.8-2.2×10⁶ cells/mL for all 4 harvests (passages).

Bioreactors 3 and 4, C1F-P2L2 cells cultivated with no exogenously provided insulin and transferrin, at the first passage reached a VCDs of about 1.9×10⁶ cells/mL. At passage 2, the VCD decreased to about 1.4×10⁶ cells/mL and at passage 3, the VCD was about 1.0×10⁶ cells/mL. At passage 4, the VCD was about 0.7×10⁶ cells/mL. The VCDs of the non-weaned C1F-P2L2 cells cultivated without the addition of insulin and transferrin decreased at every passage showing that the C1F-P2L2 cells are incapable of sustained growth in the absence of insulin. In other words, C1F-P2L2 cells cultivated in the absence of exogenously provided insulin are not viable and cannot be used for the production of cultivated meat. See FIG. 3A.

Bioreactors 5 and 6, C1F-P3 cells adapted for growth in media without insulin and transferrin showed consistent cell growth of at least 1.2×10⁶ cells/mL by day 3, with similar doubling time to the positive control cells (Bioreactors 1 and 2). Note that in FIG. 3B, the VCDs of the positive control Bioreactors 1 and 2 were about equivalent to the VCDs of the insulin and transferrin weaned C1F-P3 cells.

This Example demonstrates that C1F-P3 cells (in bioreactors 5 & 6) are well adapted to grow in media lacking insulin and transferrin.

### Example 7. Bovine Kidney Cell Line Capable of Suspension, Serum-Free, and Single Cell Culture

The bovine kidney cell described herein is of MDBK parentage. The original MDBK cell line was derived from a kidney of an apparently normal, adult steer (male), originally described, and characterized for spontaneous immortality, Feb. 18, 1957, by S. H. Madin and N. B. Darby and the established cell line was deposited with the American Type Culture Collection (ATCC) in July 1967 at passage 96. MDBK can be found for commercial purchase and research use from multiple depositories, including ATCC, European Collection of Authenticated Cell Cultures (ECACC), Riken Cell Bank (Japan), German Collection of Microorganisms and Cell Cultures (DSMZ, Germany), and JCRB Cell Bank (Japan).

The bovine kidney cell line used herein was commercially obtained as an ampoule of frozen cells. The utilized cell line originated from the MDBK cell line, modified from the parental cells by adaptation to fetal bovine serum (FBS)-independent culture (“serum-free”) and designated as MDBK-NST. MDBK-NST was deposited in the Riken Cell Bank in 2003 and was derived from the Riken Cell Bank MDBK deposit, originally deposited in 1987. The Riken Cell Bank expressly recommends MDBK-NST use for cell culture under adherent expansion and with the use of a media formulation comprising MEM supplemented with Bacto Peptone and BES. Although serum-free, the recommended Bacto Peptone supplement is an animal-origin enzymatic digest of bovine and porcine animal proteins. MDBK-NST cells used as the starting material for this experimental work were derived from Riken Cell Bank RCB1859.

### Growth Medium Preparation

The MDBK-NST cell line was adapted to serum-free culture by Riken Cell Bank, using Bacto Peptone as a growth supplement in lieu of FBS. Bacto Peptone is an animal origin enzymatic digest of bovine and porcine animal proteins. For MDBK-NST maintenance in adherent culture, the growth medium recommended by Riken Cell Bank was used, prepared according to Table 9. The osmolality of the complete culture medium was between 260-320 mOsm/Kg H₂O and the pH was between 7.2-8.0.

TABLE 9
Composition of complete MEM-bactopeptone growth media.

| Component | Concentration |
|---|---|
| MEM | As is (liquid basal media) |
| Bacto Peptone stock | 5.95 g/L |
| BES stock | 2.13 g/L |

Bacto Peptone and BES stocks were diluted in room temperature culture-grade water and dissolved by mixing before being added to the MEM basal media. The complete media was sterilized by filtration through 0.2 μm-filter prior to use in cell culture and stored at 4° C. with expiration set to one month after preparation.

### Parental Cell Initiation

A single vial containing MDBK-NST cells obtained directly from Riken Cell Bank, regarded as parental cells relative to our own subsequent expansion and banking, was thawed in-house to be used for cell line expansion. The contents of the cryovial were seeded into adherent culture targeting a inoculum density of 40,000 cell/cm², using complete MEM-bactopeptone growth medium for recovery from thaw and subsequent expansion by passaging. The cells were cultured under static incubation at 37° C., 5% CO₂ in a humidified (70-80%) atmosphere and were passaged upon reaching 70-90% confluency. Upon thaw from the parental vial, MDBK-NST were internally designated as B1E cells.

### Cell Passaging

B1E cells were routinely observed by microscopy and demonstrated epithelial-like morphology in adherent culture, characterized by polygonal shape with clear, sharp boundaries between neighbording cells. Subculture (passaging) was carried out when cells reached 70-90% confluency.

After initial culture, B1E had proliferated to 80% confluence within 5 days. The supernatant was aspirated and cultures in each flask were washed twice with Dulbecco's phosphate-buffered saline (DPBS, Gibco). Cells were enzymatically dissociated from the culture surface by incubation with TrypLE Express (Gibco) for 5-10 minutes. The sides of the flasks were tapped gently to completely release cells from the culture surface and the cultures were observed under the microscope to confirm complete dissociation. The enzymatic reaction was stopped by the addition of pre-warmed growth media (at least 5-fold more volume than the trypsinization agent). The single cell suspension was transferred to a 50 mL conical tube and cells were collected by centrifugation.

The harvested cells recovered from this first step of expansion were designated p1 (passage 1), where the initial culture from frozen was p0. A portion of B1E p1 were resuspended in pre-warmed growth medium to achieve a target inoculation density of 40,000 cells/cm² and seeded onto a tissue culture flask.

All subsequent adherent cell passaging was conducted according to the methods described for initial B1E culture.

### Cell Counting

Cell counts were preformed using Vi-Cell XR Cell Viability Analyzer (Beckman Coulter) to quantify cell density and viability. The single cell suspension was sampled directly for counting following enzymatic dissociation. The sample volume was immediately processed by the Vi-Cell XR to measure total cell density, viability, and viable cell density. The cell counting program on the Vi-Cell XR was selected for the most accurate counting of individual live cells. Based on cell counting results, population doubling time (PDT) and population doubling level (PDL) were calculated according to the following formulae, considering PDL of 0 for cells at p0:

$$PDT = t * log 10 ( 2 ) / [ ( log 10 ( n / n 0 ) ]$$

$$PDL = 3.32 [ log 10 ( n / n 0 ) ]$$

where t=time in culture, n=final cell number and n0=number of cells seeded.

Passage-to-passage growth was reported as “Cell Expansion”, quantified as the total viable cells seeded and harvested:

Total viable cells seeded (estimated): 40,000 cells/cm²*flask surface area (cm³).

Total viable cells harvested (measured):

Viable cell density (VCD, cell/mL)*working volume (mL).

### Cryopreservation

Research cell banks (RCB) were generated at each passage over B1E expansion from the parental (p0) vial. After enzymatic harvest, the volume of cell suspension that contained the desired number of cells for banking was transferred to 50 mL conical tubes and centrifuged to collect the cells as a pellet. The cell pellets were resuspended with cryopreservation solution (CELLBANKER-2, Zenoaq) that had been pre-chilled to 4° C. The volume of cryopreservation solution used achieved a final cell density of 1-10 million cells/mL. A total of 1-15 million cells were aliquot into each cryovial. Cryovials were stored in isopropanol chambers to freeze at a rate of −1° C./minute from 4° C. to −80° C. After a 24-hour freezing period in −80° C., the RCB was transferred and stored in a vapor phase liquid nitrogen storage system.

### Cell Line Expansion

B1E cells were expanded from the parental (p0) vial on T75 or T175 tissue culture flasks at a seeding density of 40,000 cells/cm² in 10 mL or 25 mL of MEM-bactopeptone media, respectively. Cells were passaged when cultures reached 70-90% confluency, approximately every 3 to 4 days.

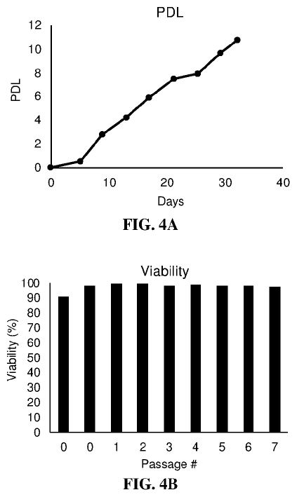

B1E cells under adherent expansion reached around 10 population doublings over 30 days with viabilities consistently above 80% under the conditions described above. The results are shown in FIG. 4.

### Adaptation of B1E Cells to Suspension

Riken Cell Bank only recommends the use of commercial MDBK-NST cells for adherent culture maintained in MEM-bactopeptone media previously described. However, our criteria for a lead beef cell line candidate to be used for process and product development requires the cell line be capable of suspension culture to maximize efficiency for large scale production.

To begin adapting B1E to suspension culture, a portion of B1E cells were transferred to suspension conditions within 2-10 passages of the parental cell adherent expansion. At the time of B1E enzymatic harvest, a portion of single cell suspension containing 20-50 million cells was collected into a 50 mL conical tube and pelleted by centrifugation. The supernatant was discarded, and the cell pellet was resuspended in pre-warmed suspension medium and seeded into a Erlenmeyer shake flask designed for suspension cultivation, targeting an inoculum density of 0.2-0.6×10⁶ cell/mL. Cells were cultured in a shaking incubator at 125 rpm rotational speed, maintained at 37° C., 5% CO₂ in a humidified (70-80%) atmosphere. Culture media used for suspension cultures was the same as that used for B1E expansion under adherence, with the addition of two supplements: 0.5% (v/v) Pluronic F-68 (Thermo Fisher), and 1.0% (v/v) Anti-clumping Agent (abbreviated ACA, Gibco). These two supplements were added to minimize cellular clumping and aggregation in suspension. Table 10 lists the complete composition of MEM-bactopeptone suspension growth media (MEMBP-S).

TABLE 10
MEM-Bactopeptone Suspension Growth Media

| Component | Concentration | |
|---|---|---|
| MEM | As is (liquid basal media) | |
| Bacto Peptone stock | 5.95 | g/L |
| BES stock | 2.13 | g/L |
| Pluronic | 0.5% | (v/v) |
| Anti-clumping Agent | 1.0% | (v/v) |

### Cell Culture Maintenance During Suspension Adaptation

B1E in suspension culture are internally designated B1E-S1. B1E-SI were maintained in suspension culture under 125 rpm rotational shaking in MEMBP-S media. Suspension culture was carried out in Erlenmeyer shake flasks ranging from 125 mL to 2.8 L volumes, selected so that the working culture volume was no more than 20-60% of the total available volume. B1E-S1 were passaged every 2-4 days to an inoculum density of 0.2-0.6×10⁶ cell/mL. Within the first several passages in suspension culture, B1E-S1 readily aggregated in culture to form multicellular clusters rather than proliferating as a single cell suspension. B1E-S1, therefore, were sub-cultured with enzymatic dissociation and spin passaging for regular maintenance. Briefly, cells were pelleted by centrifugation, and the entirety of the harvested cells were enzymatically dissociated using TrypLE. The enzymatic reaction was neutralized with the addition of pre-warmed growth media and centrifuged once more to separate the cells from the liquid fraction. The cell pellet was resuspended in pre-warmed growth medium and seeded into shake flasks. This passaging method was repeated throughout the suspension adaptation process.

### Cell Counting

For quantification of viable cell density and viability, B1E-S1 suspension cultures were sampled for cell counting. Cell samples were enzymatically dissociated for the most accurate counting of live, single cells, then immediately processed by the Vi-Cell XR automated cell counter. Based on cell counting results, PDT and PDL were calculated using previously described formulae.

### Cryopreservation

RCBs were used to preserve the progress of B1E-S1 over suspension adaptation. At passaging, the fraction of cells to be banked were enzymatically dissociated into a single cell suspension. The cells were collected as a pellet and resuspended with CELLBANKER-2 that had been pre-chilled to 4° C. The volume of cryopreservation solution used achieved a final cell density of 10 million cells/mL. A total of 5-15 million cells were aliquot into each cryovial. Cryovials were stored in isopropanol chambers to freeze at a rate of −1° C./minute from 4° C. to −80° C. After a 24-hour freezing period in −80° C., the RCB was transferred and stored in a vapor phase liquid nitrogen storage system.

### Suspension Adaptation Performance

B1E-S1 showed an immediate capacity for growth under suspension cultivation, depicted in the zig-zag trend of the VCD graph in FIG. 5. B1E-S1 indeed proliferated, though irregularly, within each 3-day suspension passage over 80 days. The tendency for aggregate formation persisted throughout continuous culture and could account for irregularities in VCD measurements at passage. Sampling of larger aggregates, for instance, would report a higher overall VCD.

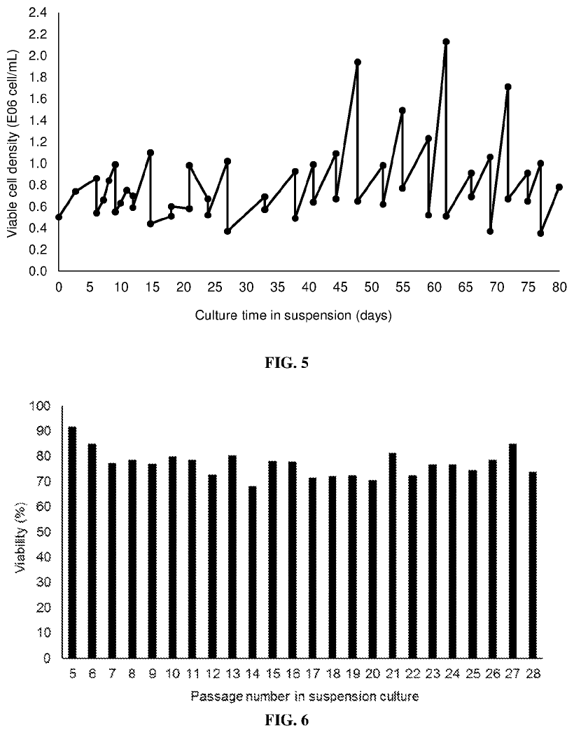

B1E-S1 multicellular aggregates contained proliferating, viable cells (see FIGS. 5 and 6). Though proliferating, cell viability was compromised during suspension adaptation, dropping from 90% viable cell population to between 60-80% viable within the initial transition period and stabilizing within this range over 80 consecutive days in culture (FIG. 6).

### Adaptation of B1E-S1 Cells to Animal-Free, Serum-Free Cultivation

MEM-bactopeptone medium was selected for the expansion of B1E and adaptation to suspension culture-based growth recommendations made by the cell line manufacturer. The primary supplement, Bacto Peptone, is a complex and undefined, animal-derived peptone subject to batch-to-batch compositional fluctuations. Adapting B1E-S1 to a chemically defined, animal-free media supports the development of a consistent, reproducible cell product at scale.

Chemically defined, serum-free media developed in-house, denoted as SFC7, was used for the adaptation of B1E-S1 away from animal-derived media components. Osmolality of SFC7 is 260-320 mOsm/Kg and pH is between 7.2-8.0.

To adapt B1E-S1 to SFC7 media cultivation, a fraction of B1E-S1 were sampled from the ongoing adaptation in MEMBP-S. The cell suspension was concentrated by centrifugation to remove residual Bacto Peptone-supplemented media, as well as enzymatically dissociated to achieve a single cell population. The cell pellet was then resuspended in pre-warmed SFC7 medium and seeded into a shake flask targeting 0.2-0.6×10⁶ cell/mL inoculum density. Due to the aggregating nature of B1E-S1, 1.0% (v/v) ACA was added to the prepared SFC7 media. Cells were cultured in the same manner as those in MEMBP-S media, with 125 rpm orbital shaking at 37° C., 5% CO₂ in a humidified (70-80%) atmosphere.

### Cell Culture Maintenance in Chemically Defined Media

B1E-S1 in SFC7 media were maintained in suspension culture according to the protocol previous described for suspension maintenance in Bacto Peptone-supplemented media. Briefly, B1E-SI were maintained in suspension culture under 125 rpm orbital shaking in SFC7 media with 1.0% (v/v) ACA. Suspension culture was carried out in shake flasks ranging from 125 mL to 2.8 L volumes with working culture volumes between 20-60% of the total available volume. B1E-S1 were passaged every 2-4 days to an inoculum density of 0.2-0.6×10⁶ cell/mL. Aggregate formation persisted in SFC7 media. Therefore, B1E-S1 were sub-cultured with enzymatic dissociation and spin passaging for regular maintenance as previously described. This passaging method was repeated for SFC7 suspension cultures in parallel with MEMBP-S cultures to compare cell performance in response to media conditions.

### Comparative Cell Performance in Chemically Defined Versus Undefined Media

B1E-S1 showed both immediate and sustained improvement in proliferation rate and viability in SFC7 media. Cells adapted to suspension cultivation in both media over time, showing overall improved growth rates and viabilities in the latter compared to the initial 20 passage groupings.

The tendency for aggregate formation persisted in animal-free, SFC7 media cultivation. Media condition does not appear to influence aggregate dynamics, except that SFC7 aggregates are slightly larger in response to increased cell growth.

### Adaptation of B1E-S1 Cells to Single Cell Growth in Suspension Culture by Size Exclusion Selection

B1E-S1 had a strong tendency to form aggregates in suspension culture. Aggregate culture poses problems for process development on a large, continuous scale. Size exclusion selection for small aggregates and single cells without enzymatic dissociation was implemented to exclude cells with aggregate tendency.

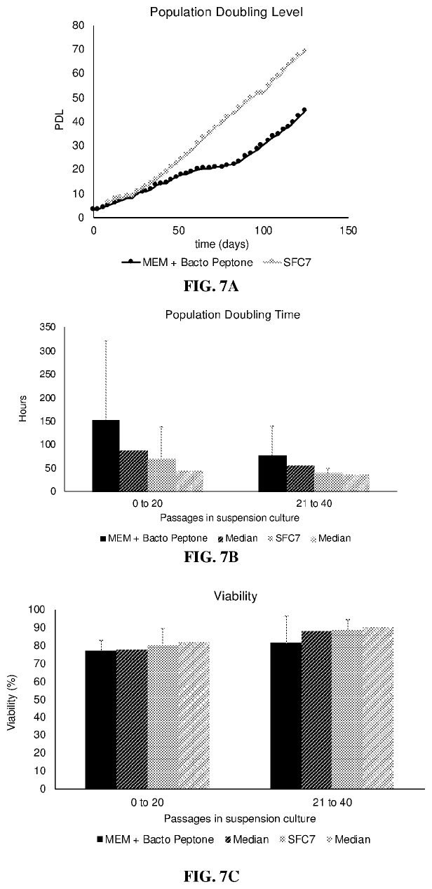

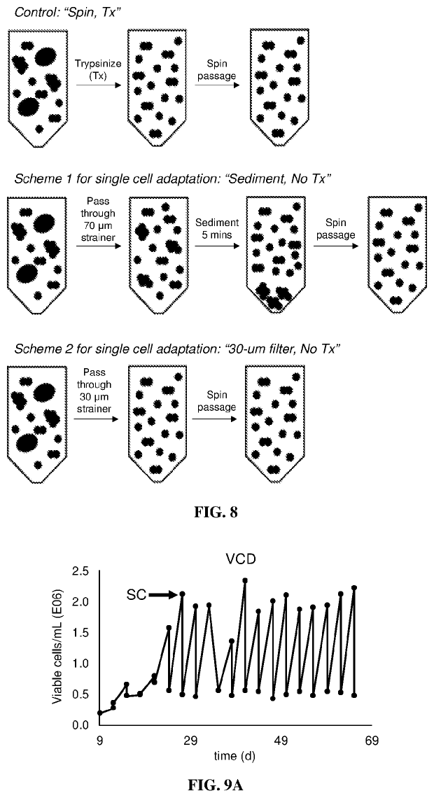

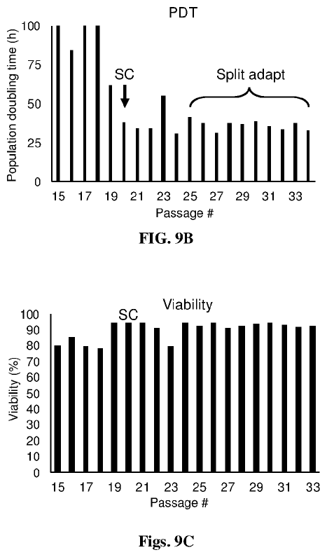

A B1E-S1 suspension-adapted culture in SFC7 medium was divided across three parallel cultures (FIG. 7):

One culture receiving the normal maintenance, control culture condition, that is spin passaging with regular enzymatic dissociation (“Spin Tx”), where “Tx” denotes the use of TypLE for enzymatic dissociation.

A second culture receiving single cell adaptation first by filtration through a sieve of pore size 40-90 μm, followed by static, gravity-driven sedimentation of the culture for 2-10 minutes and subsequent collection of the top 60-90% of the culture fraction that then received spin passaging. This process is denoted as Scheme 1, “Sediment, No Tx”, that denotes the use of sedimentation to remove larger aggregates from the cell culture without enzymatic dissociation.

The third culture receiving single cell adaptation by filtration through a sieve of pore size 10-40 μm, followed by spin passaging. This process is denoted as Scheme 2, “Filter, No Tx”, that denotes the use of size-exclusion filtration to remove aggregates greater than doublet size from the cell culture without enzymatic dissociation.

The culture receiving Scheme 2 single cell adaptation successfully generated a proliferating, single cell population within 5-20 passages of adaptation. The culture performance is shown in FIG. 8, marking the point at which the population became predominantly single cells (“SC”). From the point at which single cell adaptation is achieved, growth performance stabilizes around a 25-50 hour PDT with greater than 85% viability, even with the introduction of split passaging as opposed to spin passaging. These data suggest that a stable, serum-free, and suspension adapted single cell culture (B1E-S2) was indeed generated from the size-exclusion adaptation Scheme 1.

Conversely, B1E-S1 receiving the control spin passaging with regular enzymatic dissociation continued to form aggregates in suspension culture. Scheme 2 single cell adaptation, with a more aggressive size-exclusion approach, failed to produce a viable single cell population.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims

What is claimed:

1. A method for culturing animal cells for the production of cultivated meat, the method comprising adapting the cells to grow in a growth medium containing low-insulin or no exogenously provided insulin, low-transferrin or no-transferrin, or combinations thereof.

2. The method of claim 1, wherein the medium comprises low-insulin.

3. The method of claim 2, wherein the cells are cultivated in low-insulin growth medium that comprises less than 50 ng/L insulin.

4. The method of claim 3, wherein the cells are cultivated in low-insulin growth medium that comprises less than 10 ng/L, less than 5 ng/L, less than 1 ng/L, less than 0.5 ng/L, or less than 0.1 ng/L insulin.

5. The method of claim 1, wherein the medium comprises no exogenously provided insulin.

6. The method of any one of claims 1-5, wherein the medium comprises low-transferrin.

7. The method of claim 6, wherein the cells are cultivated in low-transferrin growth medium that comprises less than 50 ng/L transferrin.

8. The method of claim 7, wherein the cells are cultivated in low-transferrin growth medium that comprises less than 10 ng/L, less than 5 ng/L, less than 1 ng/L, less than 0.5 ng/L, or less than 0.1 ng/L transferrin.

9. The method of any one of claims 1-5, wherein the medium comprises no exogenously provided transferrin.

10. The method of any one of claim 2-4 or 6-8, wherein the cells are cultivated in a growth medium that comprises less than 50 ng/L, 40 ng/L, 30 ng/L, 20 ng/L, 10 ng/L, 5 ng/L, 3 ng/L, 1 ng/L, 0.5 ng/L, or 0.1 ng/L of each of insulin and transferrin.

11. The method of any one of claims 1-10, wherein the cell comprises the genus Gallus.

12. The method of any one of claims 1-10, wherein the cell comprises the genus Bos.

13. The method of claim 12, wherein the cell comprises an epithelial cell line.

14. The method of claim 13, wherein the epithelial cell line comprises a kidney cell line.

15. A method for culturing animals cells for the production of cultivated meat, the method comprising culturing one or more epithelial cell lines in serum-free media.

16. The method of claim 15, wherein the media is animal-free media.

17. The method of claim 15 or 16, further comprising recovering the cells and formulating the recovered cells into a paste.

18. The method of any one of claims 15-17, wherein the epithelial cell line comprises a renal epithelial cell line.

19. The method of claim 18, wherein the cell line comprises the genus Bos.

20. The method of any of one of claims 1-19, wherein the growth medium further comprises one or more of fatty acids, proteins, elements, and small molecules.

21. The method of claim 20, wherein the element comprises selenium.

22. The method of claim 20, wherein the small molecule comprises ethanolamine.

23. The method of any one of claims 1-22, wherein the cells are cultivated as an adherent culture.

24. The method of any one of claims 1-22, wherein the cells are cultivated as a suspension culture.

25. The method of claim 24, wherein the suspension culture achieves a cell density of 0.25×10⁶ cells/ml, 0.5×10⁶ cells/ml and 1.0×10⁶ cells/ml, between 1.0×10⁶ cells/ml and 2.0×10⁶ cells/ml, between 2.0×10⁶ cells/ml and 3.0×10⁶ cells/ml, between 3.0×10⁶ cells/ml and 4.0×10⁶ cells/ml, between 4.0×10⁶ cells/ml and 5.0×10⁶ cells/ml, between 5.0×10⁶ cells/ml and 6.0×10⁶ cells/ml, between 6.0×10⁶ cells/ml and 7.0×10⁶ cells/ml, between 7.0×10⁶ cells/ml and 8.0×10⁶ cells/ml, between 8.0×10⁶ cells/ml and 9.0×10⁶ cells/ml, between 9.0×10⁶ cells/ml and 10×10⁶ cells/ml, between 10×10⁶ cells/ml and 15.0×10⁶ cells/ml, between 15×10⁶ cells/ml and 20×10⁶ cells/ml, between 20×10⁶ cells/ml and 25×10⁶ cells/ml, between 25×10⁶ cells/ml and 30×10⁶ cells/ml, between 30×10⁶ cells/ml and 35×10⁶ cells/ml, between 35×10⁶ cells/ml and 40×10⁶ cells/ml, between 40×10⁶ cells/ml and 45×10⁶ cells/ml, between 45×10⁶ cells/ml and 50×10⁶ cells/ml, between 50×10⁶ cells/ml and 55×10⁶ cells/ml, between 55×10⁶ cells/ml and 60×10⁶ cells/ml, between 60×10⁶ cells/ml and 65×10⁶ cells/ml, between 70×10⁶ cells/ml and 75×10⁶ cells/ml, between 75×10⁶ cells/ml and 80×10⁶ cells/ml, between 85×10⁶ cells/ml and 90×10⁶ cells/ml, between 90×10⁶ cells/ml and 95×10⁶ cells/ml, between 95×10⁶ cells/ml and 100×10⁶ cells/ml, between 100×10⁶ cells/ml and 125×10⁶ cells/ml, or between 125×10⁶ cells/ml and 150×10⁶ cells/ml.

26. A food product comprising animals cells prepared by the method of any one of claims 1-25.

27. A cultivated animal cell, wherein the cell grows in growth medium that comprises low-insulin or no exogenously provided insulin.

28. The cell of claim 27, wherein the cell is grows in growth medium that comprises no exogenously provided insulin.

29. A cultivated animal cell, wherein the cell grows in growth medium that comprises low-transferrin or no exogenously provided transferrin.

30. The cell of claim 29, wherein the cell grows in growth medium that comprises no exogenously provided transferrin.

31. The cell of any one of claims 27-30, wherein the cell grows in growth medium that comprises low-insulin or no exogenously provided insulin, and low-transferrin or no exogenously provided transferrin.

32. The cell of any one of claims 27-31, wherein the cell is cultivated in low-insulin growth medium that comprises less than 50 ng/L insulin.

33. The cell of claim 32, wherein the cell is cultivated in low-insulin growth medium that comprises less than 10 ng/L, less than 5 ng/L, less than 1 ng/L, less than 0.5 ng/L, or less than 0.1 ng/L insulin.

34. The cell of any one of claims 27-33, wherein the cell is cultivated in low-transferrin growth medium that comprises less than 50 ng/L transferrin.

35. The cell of claim 34, wherein the cell is cultivated low-transferrin growth medium that comprises less than 10 ng/L, less than 5 ng/L, less than 1 ng/L, less than 0.5 ng/L, or less than 0.1 ng/L transferrin

36. The cell of any one of claims 27-35, wherein the cell is cultivated in a growth medium that comprises less than 50 ng/L insulin and less than 50 ng/L transferrin.

37. The cell of any one of claims 27-36, wherein the cell orginates from an animal of the genus Gallus or the genus Bos.

38. The cell of any one of claims 27-37, wherein the cell is selected from the group consisting of fibroblasts, muscle cells, myosatellite cells, myoblasts, pre-adipocytes, adipocytes, epithelial cells, lung cells, intestinal cells, and embryonic cells.

39. The cell of any one of claims 27-38, wherein the cell grows in low or no amounts of direct growth factors, animal serum, animal components, and combinations thereof.
